SEQUENCE LISTING (7 sequences)

SEQ ID NO: 1 - protein, 616 aa, Plasmodium Falciparum
GRQTSSNNEVLSNCREKRKGMKWDCKKKNDRSNYVCIPDRRIQLCIVNLAIIKTYTKETMKDHFIEASKKESQLLLKKNDNKYNSKFCNDLKNSFLDYGHLAMGNDMDFGGYSTKAENKIQEVFKGAHGEISEHKIKNFRKKWWNEFREKLWEAMLSEHKNNINNCKNIPQEELQITQWIKEWHGEFLLERDNRAKLPKSKCKNNALYEACEKECIDPCMKYRDWIIRSKFEWHTLSKEYETQKVPKENAENYLIKISEQKNDAKVSLLLNNCDAEYSKYCDCKHTTTLVKSVLNGNDNTIKEKREHIDLDDFSKFGCDKNSVDTNTKVWECKNPYKLSTKDVCVPPRRQELCLGNIDRIYDKNLLMIKEHILAIAIYESRILKRKYKNKDDKEVCKIINKTFADIRDIIGGTDYWNDLSNRKLVGKINTNSNYVHRNKKNDKLFRDEWWKVIKKDVWNVISWVFKDKTVCKEDDIENIPQFFRWFSEWGDDYCQDKTKMIETLKVECKEKPCEDDNCKSKCNSYKEWISKKKEEYNKQAKQYQEYQKGNNYKMYSEFKSIKPEVYLKKYSEKCSNLNFEDEFKEELHSDYKNKCTMCPEVKDVPISIIRNNEQTS

SEQ ID NO: 2 - protein, 616 aa, Plasmodium Falciparum
QYTFIQKRTHLFACGIKRKSIKWICRENSEKITVCVPDRKIQLCVANFLNSRLETMEKFKEIFLISVNTEAKLLYNKNEGKDPSIFCNELRNSFSDFRSSFIGDDMDFGGNTDRVKGYINTKFSDYYKEKNVEKLNNIKKEWWEKNKANLWNHMIVNHKGNISKECAIIPAEEPQINLWIKEWNENFLMEKKRLFLNIKDKCVENKKYEACFGGCRLPCSSYTSFMKKSKTQMEVLTNLYKKKNSGVDKNNFLNDLFKKNNKNDLDDFFKNEKEYDDLCDCRYTATIIKSFLNGPAKNDVDIASQINVNDLRGFGCNYKSNNEKSWNCTGTFTNKFPGTCEPPRRQTLCLGRTYLLHRGHEEDYKEHLLGASIYEAQLLKYKYKEKDENALCSIIQNSYADLADIIKGSDIIKDYYGKKMEENLNKVNKDKKRNEESLKIFREKWWDENKENVWKVMSAVLKNKETCKDYDKFQKIPQFLRWFKEWGDDFCEKRKEKIYSFESFKVECKKKDCDENTCKNKCSEYKKWIDLKKSEYEKQVDKYTKDKNKKMYDNIDEVKNKEANVYLKEKSKECKDVNFDDKIFNESPNEYEDMCKKCDEIKYLNEIKYPKTKHDI

SEQ ID NO: 3 - protein, 643 aa, Plasmodium Falciparum
NDFEKNKFIDTYSLYECGKKIKEMKWICTDNQFKSNNLCAPIRRIQLCIVNIILFSENENEYIYKNDSINNKFKENILKAVKLESNLLVQKHNNEYNSKLCDDIRWSFLDYGDIIIGRDLIYKNNTDYIKEQFKKIFNNEYNNNELNDELNNELNDEKNIKLRKEWWEKYKEDIWEEMTKEHNDKFIEKCKYFAKDEPQIVRWIEEWSKQFLDEKNYMLFTLRNTYNEMNIIHENNCKQYNKWVQNRKKEWTFLSNEFNKIFPERNVQIHISNIFKEYKENNVDIIFGTLNYEYNNFCKEKPELVSAAKYNLKAPNAKSPRIYKSKEHEESSVFGCKTKISKVKKKWNCYSNNKVTKPEGVCGPPRRQQLCLGYIFLIRDGNEEGLKDHINKAANYEAMHLKEKYENAGGDKICNAILGSYADIGDIVRGLDVWRDINTNKLSEKFQKIFMGGGNSRKKQNDNNERNKWWEKQRNLIWSSMVKHIPKGKTCKRHNNFEKIPQFLRWLKEWGDEFCEEMGTEVKQLEKICENKNCSEKKCKNACSSYEKWIKERKNEYNLQSKKFDSDKKLNKKNNLYNKFEDSKAYLRSESKQCSNIEFNDETFTFPNKYKEACMVCENPSSSKALKPIKTNVFPIEESKKSE

SEQ ID NO: 4 - protein, 639 aa, Plasmodium Falciparum
GEYLNRNSFVQRSYIRGCKGKRSTHTWICENKGNNNICIPDRRVQLCITALQDLKNSGSETTDRKLLRDKVFDSAMYETDLLWNKYGFRGFDDFCDDVKNSYLDYKDVIFGTDLDKNNISKLVEESLKRFFKKDSSVLNPTAWWRRYGTRLWKTMIQPYAHLGCRKPDENEPQINRWILEWGKYNCRLMKEKEKLLTGECSVNRKKSDCSTGCNNECYTYRSLINRQRYEVSILGKKYIKVVRYTIFRRKIVQPDNALDFLKLNCSECKDIDFKPFFEFEYGKYEEKCMCQSYIDLKIQFKNIDICSFNAQTDTVSSDKRFCLEKKEFKPWQCDKNSFETVHHKGVCVSPRRQGFCLGNLNYLLNDDIYNVHNSQLLIEIIMASKQEGKLLWKKHGTILDNQNACKYINDSYVDYKDIVIGNDLWNDNNSIKVQNNLNLIFERNFGYKVGRNKLFKTIKELKNVWWILNRNKVWESMRCGIDEVDQRRKTCERIDELENMPQFFRWFSQWAHFFCKEKEYWELKLNDKCTGNNGKSLCQDKTCQNVCTNMNYWTYTRKLAYEIQSVKYDKDRKLFSLAKDKNVTTFLKENAKNCSNIDFTKIFDQLDKLFKERCSCMDTQVLEVKNKEMLSIDSNSEDA

SEQ ID NO: 5 - protein, 646 aa, Plasmodium Falciparum
VIIIIRIILIVRIHFLLWINLSIDVKRELKENEIGHAMIRIQRNQIYVFLIEEYNYVQVILNFLLMILLKRNLKRNFWQHKKKGVYYLKSLVKNIMKNFVLINGVMVITEILKVLTWKDLVDQLRLNLKIEYLKGIIRNLRKIGKNGGKKLVLIYGMLKNIKRNFQRMKNVQQINLMNHKLIDGWNGLMIQIIIKIYGIINWMILKINVIEFVQKKEICKSECKKYKAWIDKKNNDFTILSEIYLKYNKKSSLYKTAFEYLKQKWDKYKELNFSSIFDQLNAKYYNKCICQNNKIENNALYVKIEDICNNTKVKSIYGELYCKEKGNDKIWQCINEHIKDFPDVCGPPRRQQLCLGNLDKDEFKNVNDLKKFLNEIILGIRDEGKFLIEKYRKNMHENMYLDERACKYLNYSFDDYKNIILGKDMWRDPNSIKTENILKGNFEGIKANIVSMYPSYADLSLDEFRKHWWDQNKKQLWEAISCEFYKGNHTGVCLMEDDNDNQYLHWFREWKNDFCIDKLKWNDVIKEPCIDKKVKSPKPSENPSDVATVCNKSCTDYDKWIINKRKEYKMQSSKYKRDRSLFNNVIQNLKPWEYLSMKCTECTCNLDTQTFVYPYKGYEDICKSTVKPYDPEDIKDEEFNEPSLNV

SEQ ID NO: 6 - DNA, 1848 bp, Plasmodium Falciparum
ggccgccagacctcctccaacaacgaggtgctgtccaactgccgcgagaagcgcaagggcatgaagtgggactgcaagaagaagaacgaccgctccaactacgtgtgcatccccgaccgccgcatccagctgtgcatcgtgaacctggccatcatcaagacctacaccaaggagaccatgaaggaccacttcatcgaggcctccaagaaggagtcccagctgctgctgaagaagaacgacaacaagtacaactccaagttctgcaacgacctgaagaactccttcctggactacggccacctggccatgggcaacgacatggacttcggcggctactccaccaaggccgagaacaagatccaggaggtgttcaagggcgcccacggcgagatctccgagcacaagatcaagaacttccgcaagaagtggtggaacgagttccgcgagaagctgtgggaggccatgctgtccgagcacaagaacaacatcaacaactgcaagaacatcccccaggaggagctgcagatcacccagtggatcaaggagtggcacggcgagttcctgctggagcgcgacaaccgcgccaagctgcccaagtccaagtgcaagaacaacgccctgtacgaggcctgcgagaaggagtgcatcgacccctgcatgaagtaccgcgactggatcatccgctccaagttcgagtggcacaccctgtccaaggagtacgagacccagaaggtgcccaaggagaacgccgagaactacctgatcaagatctccgagaacaagaacgacgccaaggtgtccctgctgctgaacaactgcgacgccgagtactccaagtactgcgactgcaagcacaccaccaccctggtgaagtccgtgctgaacggcaacgacaacaccatcaaggagaagcgcgagcacatcgacctggacgacttctccaagttcggctgcgacaagaactccgtggacaccaacaccaaggtgtgggagtgcaagaagccctacaagctgtccaccaaggacgtgtgcgtgcccccccgccgccaggagctgtgcctgggcaacatcgaccgcatctacgacaagaacctgctgatgatcaaggagcacatcctggccatcgccatctacgagtcccgcatcctgaagcgcaagtacaagaacaaggacgacaaggaggtgtgcaagatcatccagaagaccttcgccgacatccgcgacatcatcggcggcaccgactactggaacgacctgtccaaccgcaagctggtgggcaagatcaacaccaactccaactacgtgcaccgcaacaagcagaacgacaagctgttccgcgacgagtggtggaaggtgatcaagaaggacgtgtggaacgtgatctcctgggtgttcaaggacaagaccgtgtgcaaggaggacgacatcgagaacatcccccagttcttccgctggttctccgagtggggcgacgactactgccaggacaagaccaagatgatcgagaccctgaaggtggagtgcaaggagaagccctgcgaggacgacaactgcaagcgcaagtgcaactcctacaaggagtggatctccaagaagaaggaggagtacaacaagcaggccaagcagtaccaggagtaccagaagggcaacaactacaagatgtactccgagttcaagtccatcaagcccgaggtgtacctgaagaagtactccgagaagtgctccaacctgaacttcgaggacgagttcaaggaggagctgcactccgactacaagaacaagtgcaccatgtgccccgaggtgaaggacgtgcccatctccatcatccgcaacaacgagcagacctcc

SEQ ID NO: 7 - protein, 616 aa, Plasmodium Falciparum
GRQTSSNNEVLSNCREKRKGMKWDCKKKNDRSNYVCIPDRRIQLCIVNLAIIKTYTKETMKDHFIEASKKESQLLLKKNDNKYNSKFCNDLKNSFLDYGHLAMGNDMDFGGYSTKAENKIQEVFKGAHGEISEHKIKNFRKKWWNEFREKLWEAMLSEHKNNINNCKNIPQEELQITQWIKEWHGEFLLERDNRAKLPKSKCKNNALYEACEKECIDPCMKYRDWIIRSKFEWHTLSKEYETQKVPKENAENYLIKISENKNDAKVSLLLNNCDAEYSKYCDCKHTTTLVKSVLNGNDNTIKEKREHIDLDDFSKFGCDKNSVDTNTKVWECKKPYKLSTKDVCVPPRRQELCLGNIDRIYDKNLLMIKEHILAIAIYESRILKRKYKNKDDKEVCKIIQKTFADIRDIIGGTDYWNDLSNRKLVGKINTNSNYVHRNKQNDKLFRDEWWKVIKKDVWNVISWVFKDKTVCKEDDIENIPQFFRWFSEWGDDYCQDKTKMIETLKVECKEKPCEDDNCKRKCNSYKEWISKKKEEYNKQAKQYQEYQKGNNYKMYSEFKSIKPEVYLKKYSEKCSNLNFEDEFKEELHSDYKNKCTMCPEVKDVPISIIRNNEQTS

(19) United States
(12) Patent Application Publication
(10) Pub. No.: US 2007/0043208 A1
(43) Pub. Date: Feb. 22, 2007

(54) Crystal structure of erythrocyte binding domain of EBA-175

(76) Inventors: Leemor Joshua-Tor, Huntington, NY (US); Niraj H. Tolia, Cold Spring Harbor, NY (US); B. Kim Lee Sim, Gaithersburg, MD (US)

Correspondence Address: Leopold Presser;Scully, Scott, Murphy & Presser, Suite 300, 400 Garden City Plaza, Garden City, NY, 11530, US

(21) Appl. No.: 11/183,666

(22) Filed: Jul. 18, 2005

Related U.S. Application Data
(63) Continuation-in-part of application No. 10/861,615, filed on Jun. 4, 2004.
(60) Provisional application No. 60/476,489, filed on Jun. 6, 2003.

(51) Int. Cl. A61K 38/17; C07K 14/74; G06F 19/00; G01N 33/48; G01N 33/50

(52) U.S. Cl. 530/350; 702/19

(57) ABSTRACT

The present invention provides three-dimensional structural information for region II of the erythrocyte binding antigen 175 (EBA-175). Specifically, the present invention provides three-dimensional structural information of erythrocytic receptor binding sites of EBA-175 RII. The three-dimensional structural information is useful in drug design aimed at blocking receptor interaction with EBA-175. Methods for drug design and methods for identifying compounds binding to EBA-175 RII are also provided.

### CROSS REFERENCE TO RELATED APPLICATIONS

The present application is a continuation-in-part of U.S. application Ser. No. 10/861,615 filed Jun. 4, 2004, now pending, herein incorporated by reference, which claims the benefit of U.S. Provisional Application No. 60/476,489 filed Jun. 6, 2003.

### GOVERNMENT SUPPORT

This invention was made in part with Government support under Grant Number NIH-NIAID-DMID-00-18 Grant No. AI05421 awarded by the National Institutes of Health. The United States government may have certain rights in this invention.

### BACKGROUND OF THE INVENTION

Malaria disease accounts for more than three million deaths per year, with more than 2 million deaths being children under the age of five. The disease is manifested by parasites of the genus Plasmodium, of which Plasmodium falciparum is the most prevalent. The female Anopheles mosquito (the vector) is responsible for transmitting the parasite. Upon inoculation, by a bite of the vector, the malarial parasite in the sporozite stage first invades hepatocytes. Five days later, the parasite develops into a mature liver stage schizont with thousands of merozites. On rupture of the hepatocyte the merozites are released into the blood stream and can invade erythrocytes, resulting in the erythrocytic stages of the parasite which is responsible for the clinical manifestation. Thus, the parasite is primarily intracellular during an infection. Two important exceptions are the sporozite and merozite stage.

In order to invade erythrocytes, the parasite must first recognize and bind to erythrocytic surface ligands. Initial studies revealed that invasion was dependent on sialic acid found on glycophorins (Miller, L. H. et al., J. Exp. Med., 146 (1):277-281 (1977); Pasvol, G. et al., Nature, 297 (5861):64-66 (1982); Perkins, M., J. Cell Biol., 90 (3):563-567 (1981); Breuer, W. V. et al., Biochim. Biophys. Acta, 755 (2):263-271 (1983); and Hadley, T. J. et al., J. Clin. Invest., 80 (4):1190-1193 (1987)), the major glycoprotein on erythrocytes. Both neuraminidase treated erythrocytes and erythrocytes lacking glycophorins are resistant to invasion (reviewed in Hadley, T. J. et al., Tranfus Med. Rev., 5 (2):108-122 (1991)).

Identification of a 175 kDa protein from P. falciparum cultures, that bound human erythrocytes but not human erythrocytes treated with neuraminidase, revealed the merozite ligand termed erythrocyte binding antigen 175 (EBA-175) (Camus, D. et al., Science, 230 (4725):553-556 (1985)). Binding of EBA-175 is specific to sialic acids-2,3 on O-linked tetrasaccharides (Orlandi, P. A. et al., J. Cell. Biol., 116 (4):901-909 (1992)). Conversion of the sialic acid on mouse erythrocytes to the human form enhances the binding of EBA-175 to mouse erythrocytes (Klotz, F. W. et al., Mol. Biochem. Parasitol., 51 (1):49-54 (1992)). Antibodies raised against a peptide from EBA-175 that block binding to EBA-175 to erythrocytes inhibit merozite invasion into erythrocytes (Sim, B. K. et al., J. Cell Biol., 111 (5 Pt 1): 1877-1884 (1990)).

Cloning of the gene encoding EBA-175 revealed that its gene structure was similar to the Duffy-binding proteins of P. vivax and P. knowlesi (Adams, J. H. et al., Proc. Natl. Acad. Sci. USA, 89 (15):7085-7089 (1992)). The Duffy-binding proteins and EBA-175 have analogous functional roles. It is believed that these proteins are evolutionarily conserved receptor binding proteins.

Dissection of EBA-175, based on its homology to the Duffy-binding proteins, into six regions (I-VI) revealed that only region II (RII) of EBA-175 conferred the ability of COS cells to bind erythrocytes forming a rosette (Sim, B. K. et al., Science, 264 (5167):1941-1944 (1994); and Chitnis, C. E. et al., J. Exp. Med., 180 (2):497-506 (1994)). In addition, the binding of RII to normal, mutant and neuraminidase treated erythrocytes exhibited identical properties as the full-length EBA-175. That is, RII was unable to bind erythrocytes lacking glycophorin A or sialic acid. Region II of the Duffy-binding proteins is also responsible for recognition of the Duffy-antigen, providing further evidence of the role of this region in receptor binding.

RII is a 73 kDa domain (616 amino acid fragment of EBA-175) that contains a duplicated Cys-rich region (termed F1 and F2), each homologous to the Duffy binding protein of P. vivax, a single cysteine rich domain. The cysteine motifs (Duffy binding like, DBL domains) allowed the classification of EBA-175 and the Duffy binding protein to be grouped as a family of erythrocyte-binding like (ebl) proteins (Adams, J. H. et al., Proc. Natl. Acad. Sci. USA, 89:7085-7089 (1992)), all of which are believed to be involved in erythrocyte invasion and whose defining features are the presence of an RII-like region (one or two DBL domains), a C-terminal cys rich region (region VI), and a type-I transmembrane like domain. Subsequently, the var genes, a large family of genes that encode antigenically variant proteins that include PfEMP1, responsible for cytoadherence of parasitized erythrocytes to endothelium causing the most fatal form of malaria disease, were also found to contain several copies of DBL domains (Baruch, D. I. et al., Cell, 82: 77-87 (1995); Su, X. Z. et al., Cell, 82: 89-100 (1995); Smith, J. D. et al., Cell, 82:101-110 (1995)).

RII is a highly basic protein with a calculated pI of 8.78. This highly basic nature is believed to play a role in binding the negatively charged sialic acid. Alignment of RIIs from P. falciparum (F 1 and F2), P. knowlesi and P. vivax revealed the high conservation of a number of cysteine, tryptophan and tyrosine residues (Adams, J. H. et al., Proc. Natl. Acad. Sci. USA, 89 (15):7085-7089 (1992)). It is possible that these residues are directly implicated in ligand recognition. Antibodies that inhibited merozite invasion were raised against a peptide found in region V (Sim, B. K. et al., J. Cell. Biol., 111 (5 Pt 1): 1877-1884 (1990)). It is believed that these antibodies bind to EBA-175 preventing erythrocyte binding by inducing steric hinderance rather than directly affecting the interaction sites.

Glycophorin A (GpA) is the major sialoglycoprotein found on erythrocytes. GpA is a 131 amino acid transmembrane protein that spans the membrane once and exposes its N-terminus at the extracellular surface. The C-terminal 23 amino acid transmembrane segment of GpA has been determined by NMR to form a compact helical dimer (MacKenzie, K. R. et al., Science, 276 (5309):131-133 (1997)). The N-terminus of GpA is heavily modified by O-glycosylation introducing tetrasaccharides of the composition Sia_{—}2-3Galβ1-3 (Sia_{—}2-6)GalNAc_{—}1-Ser/Thr.

A related glycoprotein, glycophorin B (GpB), is also found on erythrocytes and posses the same 11 O-glycosylation sites as GpA. GpB, however, lacks the single N-glycosylation site found in GpA. In addition, GpB differs from GpA in its amino acid sequence. Examination of the ligand on the erythrocytes involved in EBA-175 binding (Sim, B. K. et al., Science, 264 (5167):1941-1944 (1994)) indicated that erythrocytes lacking GpA but not GpB were unable to bind EBA-175. Furthermore, soluble GpA inhibited binding of EBA-175 to erythrocytes, while soluble GpB did not. Treatment of erythrocytes with N-glycanase, which results in removal of N-linked oligosaccharides but not O-linked oligosaccharides, had no effect on EBA-175 binding indicating that the difference in N-linked glycosylation was not sufficient to explain the difference in binding.

Further dissection of GpA revealed that the extracellular domain (a glycopeptide including residues 1-64 of GpA) inhibited binding to the same extent as GpA. However, a mixture of glycopeptide 1-34 and glycopeptide 35-64 was unable to inhibit EBA-175 binding, indicating the requirement for the full-length extracellular domain. These results together suggest the role of the amino acid sequence difference between GpB and GpA in EBA-175 binding. It is uncertain whether there is a direct contact between EBA-175 and the protein of glycophorin A or whether the protein chain allows for a unique conformation of the carbohydrate portion of GpA facilitating binding. Nevertheless the erythrocytic ligand for EBA-175 believed to be GpA.

Knowledge of the structure of EBA-175 region II would facilitate the development and design of therapeutic drugs and vaccines that prevent EBA-175 from binding to erythrocytes and halt merozite invasion.

### SUMMARY OF THE INVENTION

The present invention identifies the first crystal structure of the Duffy-binding like (DBL) domains from the erythrocyte-binding like (ebl) family of proteins, providing a glimpse into the mechanism of erythrocyte invasion by the Plasmodium parasite and insight into a large superfamily of proteins critical in the pathogenesis and survival of the parasite. The structure provides methods for rational design of receptor blockade therapeutics and vaccines that can aid in the treatment of malaria so desperately needed.

Specifically, the present invention identifies at least one receptor binding site on a DBL domain of the ebl family proteins, particularly, erythrocyte binding antigen 175 (EBA-175) region II, that specifically binds to its erythrocytic ligand or receptor glycophorin A (GpA). In one aspect, the present invention identifies six GpA receptor/ligand binding sites on EBA-175 region II (RII) or the erythrocyte-binding domain of EBA-175. In a particular aspect, the present invention identifies a first set of receptor bind sites comprising residues N417, R422, N429, K439 and D442 of one monomer of an EBA-175 RII dimer and K28 of the other monomer (i.e., receptor binding sites 1 and 2), a second set of receptor binding sites comprising residues N550, N551, Y552, K553 and M554 from one monomer of the EBA-175 RII dimer and N33 from the other monomer (i.e., receptor binding sites 3 and 4), and a third set of receptor binding sites comprising residues T340, K341, D342, V343, Y415, Q542 and Y546 of one monomer of the RII dimer and residues K28, N29, R31 and S32 of the second monomer (i.e., receptor binding sites 5 and 6).

The present invention provides detailed three-dimensional atomic structural information for both unbound EBA-175 RII and RII-ligand complex. In one aspect, three-dimensional structural information is obtained from a crystal of EBA-175 RII (form 1) having unit cell dimensions of about a=76.96 A, b=76.96 A, c=277.33 A and alpha=beta=gamma=90 and/or a crystal of EBA-175 RII (form 2) having unit cell dimensions of about a=103.65 A, b=103.65 A, c=212.72 A and alpha=beta=gamma=90. In a particular embodiment, the crystals of form 1 form in space group P4₃2₁2 and the crystals of form 2 form in space group P4₁22.

In another aspect, the three-dimensional structural information of the EBA-175 RII protein is obtained from a crystal of an EBA-175 RII-ligand complex, which comprises the EBA-175 RII protein complexed with a ligand. For example, a crystal of an EBA-175 RII-sialylactose complex (form 3) forms in space group C222₁ and has unit cell dimensions of about a=145.74 A, b=146.21 A, c=214.74 A, and alpha=beta=gamma=90.

The present invention also contemplates crystals that form in other space groups. In one aspect, three-dimensional structural information is obtained from a crystal of EBA-175 RII that diffracts to a resolution limit of at least equal to or greater than (numerically less than) 2.3 (2.25) angstrom (A). In a particular embodiment, three-dimensional structural information is obtained from a crystal of EBA-175 RII having the structure defined by the atomic coordinates of Table 5. In a further embodiment, three-dimensional structural information is obtained from a co-crystal of an EBA-175 RII-receptor/inhibitor compound complex, which has the structure defined by the atomic coordinates of Table 7.

One aspect of the present invention provides a method for crystallizing a DBL domain protein of ebl family, such as EBA-175 RII domain, for determining the three-dimensional atomic structure of the DBL domain protein, which comprises mixing a protein solution of the DBL domain protein with a reservoir solution in any crystallization setup (preferably, but not exclusively, hanging drop, sitting drop, free liquid diffusion, batch or micro-batch), crystallizing the mixture in a closed container at a temperature of about 0° C. to about 37° C., preferably, 17° C., for a sufficient period time, e.g., several hours to weeks, preferably, 2 days, to form the crystals of the DBL domain protein, wherein the protein solution contains about 1 mg/ml to about 30 mg/ml DBL domain protein, e.g., EBA-175 RII protein, and in about 1-100 mM buffer that can hold a pH value of about 5.5 to about 9.0, and about 0-400 mM salt, e.g., NaCl or equivalent. More preferably, the protein solution contains EBA-175 RII at a concentration of about 12 or about 15 mg/ml, and further contains about 10 mM Tris-HCl and about 100 mM NaCl and has a pH value of about 7.4. The reservoir solution contains either about 0.1-0.4 M ammonium sulfate, about 0.01-0.2 M buffer that can hold a pH value of about 5.5-9.0, and about 10-35% polyethylene glycol or polyethylene glycol monomethyl ether having a molecular weight (MW) of about 1,000 to about 20,0000, or about 1.5-3.5 M ammonium sulfate, about 0.01-0.2 M buffer that can hold a pH value of about 5.5-9.0, and about 0.01-10% polyethylene glycol or polyethylene glycol monomethyl ether having a molecular weight (MW) of about 100 to about 1,000. Preferably, the reservoir solution contains either about 0.265 M ammonium sulfate, about 0.1 M sodium cacodylate at pH about 6.5 and about 29% polyethylene glycol 8000 for mixing with a protein solution of about 15 mg/ml of the protein, or about 2.6-2.8 M ammonium sulfate, about 0.1 M sodium cacodylate at pH about 6.5 and about 0.05-2% polyethylene glycol 750 monomethyl ether for mixing with a protein solution of about 12 mg/ml of the protein.

Another aspect of the present invention provides a method for co-crystallizing a DBL domain protein-ligand complex, e.g., the EBA-175 RII-ligand complex, for determining the receptor binding sites on three-dimensional atomic structure of EBA-175 RII, which comprises mixing a protein solution of DBL domain, e.g., EBA-175 RII, with a reservoir solution, and a ligand solution in any crystallization setup (preferably, but not exclusively, hanging drop, sitting drop, free liquid diffusion, batch or micro-batch), crystallizing the mixture in a closed container at a temperature of about 0° C. to about 37° C., preferably, 17° C., for a sufficient time, e.g., several hours to several weeks, preferably, 2 days, to form the crystals of the DBL domain protein, wherein the protein solution contains about 1 mg/ml to about 30 mg/ml DBL domain protein, e.g., EBA-175 RII protein, and in about 1-100 mM buffer that can hold a pH value of about 5.5.-9.0 and about 0-400 mM salt, e.g., NaCl or equivalent. More preferably, the protein solution contains EBA-175 RII at a concentration of about 12 or 15 mg/ml, and further contains about 10 mM Tris-HCl and about 100 mM NaCl and has a pH value of about 7.4. The reservoir solution contains either about 0.1-0.4 M ammonium sulfate, about 0.01-0.2 M buffer that can hold a pH value of about 5.5-9.0, and about 10-35% polyethylene glycol or polyethylene glycol monomethyl ether having a molecular weight (MW) of about 1,000 to about 20,000, or about 1.5-3.5 M ammonium sulfate, about 0.01-0.2 M buffer that can hold a pH value of about 5.5-9.0, and about 0.01-10% polyethylene glycol or polyethylene glycol monomethyl ether having a molecular weight (MW) of about 100 to about 1,000. Preferably, the reservoir solution contains either about 0.265 M ammonium sulfate, about 0.1 M sodium cacodylate at a pH value of about 6.5 and about 29% polyethylene glycol 8000 for mixing with a protein solution of about 15 mg/ml or about 2.6-2.8 M ammonium sulfate, about 0.1 M sodium cacodylate at a pH value of about 6.5 and about 0.05-2% polyethylene glycol 750 monomethyl ether for mixing with a protein solution of about 12 mg/ml. The ligand (e.g., α-2,3-sialyllactose) solution used above can have a concentration of about 1 nM to about 100 mM, preferably, the ligand solution is an α-2,3-sialyllactose solution having a concentration of about 10 mM.

The EBA-175 RII protein used for crystallization (SEQ ID NO: 7) in the present invention differs from wild-type EBA-175 RII protein, by containing mutations at five putative N-linked glycosylation sites (N3Q, S50A, S 195A, T206A, and N400Q). Such mutations avoid deleterious glycosylation of the EBA-175 RII protein and allow crystallization of the protein.

Any crystallization technique known to those skilled in the art can be employed to obtain the crystals of the present invention, including, but not limited to, vapor diffusion (either by sitting drop or hanging drop), batch crystallization or micro dialysis. In a particular aspect, the crystallization is by hanging drops using vapor diffusion method to a resolution of at least equal or greater than (numerically less than) about 2.3 (2.25) A. In another particular aspect, derivatized crystals of RII can be obtained by soaking RII crystals in an appropriate solution, preferably, saturated lithium sulfate containing either sodium dicyanoaureate or potassium tetrachloroplatinate for a sufficient period of time, preferably, 2 hours.

The three-dimensional structural information obtained from the crystals of the present invention provides a means for designing new candidate compounds which have the potential to inhibit binding of EBA-175 to glycophorin A, to erythrocytes or to both glycophorin A and erythrocytes, thereby halting and/or blocking merozite invasion into erythrocytes.

The present invention also contemplates other uses of the structural information of EBA-175 RII identified by the present inventions, including, but not limited to, protein structure prediction, vaccine design or structure-assisted drug design.

The three-dimensional structural information obtained from the crystals of the invention also provides a means for designing new candidate compounds which have the potential to affect the specificity or activity of EBA-175 in other ways. As a result, the present invention also provides computer-based methods for the rational design, identification and screening of candidate modulator compounds of EBA-175.

In one aspect, the method for the rational design, identification and/or screening of candidate modulator compounds of EBA-175 comprises the steps of: (a) providing a three-dimensional structure of EBA-175 RII; (b) providing a three-dimensional structure of a candidate molecule; and (c) fitting the structure of said candidate molecule to the structure of said EBA-175 RII. In a second aspect, the method for the rational design, identification and/or screening of candidate modulator compounds of EBA-175 comprises the steps of: (a) providing a three-dimensional structure of EBA-175 RII as defined by the atomic coordinates of Table 5 or Table 7; (b) providing a three-dimensional structure of a candidate molecule; and (c) fitting the structure of said candidate molecule to the structure of said EBA-175 RII of Table 5 or Table 7.

In another aspect, the methods for the rational design, identification and/or screening of candidate modulator compounds of EBA-175 utilizes the atomic coordinates of atoms of interest of EBA-175 RII which are in the vicinity of putative substrate and/or co-factor binding sites (regions) in order to model the pocket in which the substrate or co-factor binds. These coordinates can be used to define a space which is then screened “in silico” against a candidate modulator compound. In a particular aspect of the present invention, a computer-based method for the rational design, identification and/or screening of candidate modulator compounds of EBA-175 comprises the steps of: (a) providing atomic coordinates of a surface of a channel created by a dimer of EBA-175 RII; (b) providing a three-dimensional structure of a candidate modulator compound; and (c) fitting the structure of said candidate modulator compound to the provided coordinates. In a second embodiment, the method comprises the steps of: (a) providing atomic coordinates of a binding site of surface sulfate molecules of EBA-175 RII; (b) providing a three-dimensional structure of a candidate modulator compound; and (c) fitting the structure of said candidate modulator compound to the provided coordinates. In a third embodiment, the method comprises the step of: (a) providing atomic coordinates of one or more sialic acid binding site of EBA-175 RII; (b) providing a three-dimensional structure of a candidate modulator compound; and (c) fitting the structure of said candidate modulator compound to the provided coordinates. In a fourth embodiment, the method comprises the steps of: (a) providing atomic coordinates of an antibody binding site of EBA-175 RII; (b) providing a three-dimensional structure of a candidate modulator compound; and (c) fitting the structure of said candidate modulator compound to the provided coordinates.

After candidate modulator compounds have been designed or selected (by means including, but not limited to, in silico analysis, wet chemical methods, X-ray analysis, and NMR) by determining those which have desirable (favorable) fitting properties (e.g., strong attraction between candidate compound and EBA-175 RII), the designed or selected compounds can be screened for activity. As such, in a particular embodiment, the methods for the rational design, identification and/or screening of candidate modulator compounds of EBA-175 further comprise (d) obtaining or synthesizing said candidate inhibitor compound; and (e) contacting said candidate compound with EBA-175 RII to determine the ability of said candidate compound to interact with EBA-175 RII. In another embodiment, the methods for the rational design, identification and/or screening of candidate modulator compounds of EBA-175 further comprise (d) obtaining or synthesizing said candidate modulator compound; (e) forming a complex of EBA-175 RII and said candidate modulator compound; and (f) analyzing said complex by X-ray crystallography or NMR to determine the ability of said candidate compound to interact with EBA-175 RII. Detailed structural information can then be obtained about the binding of the candidate compound to EBA-175 RII. Adjustments can be made to the structure or functionality of the candidate compound (e.g., to improve binding to the binding site), if appropriate, based on the detailed structural information. These steps can be repeated as needed.

The invention also provides a method for the rational design, identification and/or screening of candidate modulator compounds of EBA-175 comprising the steps of: (a) using a three-dimensional structure of EBA-175 RII as defined by atomic coordinates of Table 5 or 7; (b) employing the three-dimensional structure to design or select a candidate modulator compound; (c) synthesizing and/or selecting the candidate modulator compound; and (d) determining whether the candidate compound is capable of forming a complex with EBA-175 RII or a functional variant thereof. Formation of the complex of the EBA-175 RII and candidate compound indicates that the candidate compound binds to EBA-175. Candidate compounds that bind to EBA-175 can be screened for activity (e.g., the ability to inhibit binding of EBA-175 to glycophorin A, to erythrocytes or to both glycophorin A and erythrocytes).

In a further aspect, the binding properties of a rationally designed candidate compound can be determined by a method comprising the steps of: (a) co-crystallizing a candidate compound with EBA-175 RII; (b) determining the three-dimensional structure of the EBA-175 RII-candidate compound complex co-crystal; and (c) analyzing said three-dimensional structure to determine the binding characteristics of the candidate inhibitor compound. Three-dimensional structure of the EBA-175 RII-candidate compound complex co-crystal can be determined by means including, but not limited to, molecular replacement or refinement using the three-dimensional structure of EBA-175 RII as defined by the atomic coordinates of Table 5 or Table 7, multiple isomorphous replacement (MIR) analysis, multi-wavelength anomalous dispersion or multiple anomaly dispersion (MAD), or model building and refinement.

In another aspect, the invention provides a method of detecting and/or identifying a candidate compound that binds to EBA-175 comprising the steps of: (a) from a database of candidate compounds containing docking information, sampling one or more candidate compounds and assessing whether the candidate compound(s) are capable of forming a complex with EBA-175 RII or a functional variant thereof; and (b) identifying a conformation of a candidate compound suitable for docking to said EBA-175 RII, wherein identification of said conformation of said candidate compound is indicative that said candidate compound binds to EBA-175. In one embodiment, the compound database comprises information for calculating interaction energy between said candidate compounds and EBA-175 RII. In a second embodiment, the compound database comprises information for generating one or more conformations of said candidate compounds. In another embodiment, identifying a conformation of a candidate compound suitable for docking to said EBA-175 RII comprises consideration of all possible binding modes and conformations of the candidate compound to identify a compound that optimally docks to EBA-175 RII.

The invention further provides a method of using the crystals of the invention for screening for a novel drug comprising: (a) selecting a candidate compound by performing rational drug design with the three-dimensional structure determined for the crystal; (b) contacting the candidate compound with EBA-175 RII or a functional variant thereof; and (c) detecting the binding potential of the candidate compound for EBA-175 RII or said variant, wherein the novel drug is selected based on its having a greater affinity for EBA-175 RII or said variant than that of a known drug.

In a particular embodiment, the methods of the invention involve selecting candidate modulator compounds or drugs by computationally screening a database of compounds for interaction with binding sites or regions.

In addition to de novo drug design, the present invention also relates to a structure-assisted drug design method, which preferably comprises the steps of: (a) providing a three-dimensional structure of EBA-175 RII; and (b) modifying a pre-designed or pre-selected candidate modulator compound based on the three-dimensional structure of EBA-175 RII for enhanced binding of said candidate modulator compound with EBA-175 RII. Preferably, the method may further comprise determining whether the binding between the modified candidate modulator compound and EBA-175 RII is enhanced.

The invention also provides a method of displaying a three-dimensional molecular model of EBA-175 RII on a computer system comprising (a) providing atomic coordinate data according to Table 5 or Table 7; and (b) analyzing said data using a protein-modeling algorithm.

The invention also provides a method for rational vaccine design comprising (a) providing a three-dimensional structure of EBA-175 RII; (b) determining one or more receptor binding site(s) or oligomerization interface from said three-dimensional structure of EBA-175 RII; (c) providing an amino acid sequence of a EBA-175 RII peptide which includes one or more said receptor binding site(s) and/or oligomerization interface (oligomerization/dimerization interaction site(s)); and (d) formulating said peptide of step (c) as a vaccine composition. In another embodiment, the invention provides a method for rational vaccine design comprising (a) providing a three-dimensional structure of EBA-175 RII as defined by the atomic coordinates of Table 5 or 7; (b) determining one or more receptor binding site(s) or oligomerization interface from the three-dimensional structure of EBA-175 RII; (c) providing an amino acid sequence of a EBA-175 RII peptide which includes one or more said receptor binding site(s) and/or oligomerization interface; and (d) formulating said peptide of step (c) as a vaccine composition. EBA-175 RII peptides which include one or more said receptor binding site(s) and/or oligomerization interface (oligomerization/dimerization interaction site(s)) include EBA-175 RII peptides which include one or more receptor binding site(s), EBA-175 RII peptides which include one or more oligomerization/dimerization interaction site(s), and EBA-175 RII peptides which include one or more receptor binding site(s) and one or more oligomerization/dimerization site(s).

This invention further pertains to novel compounds identified by the above-described methods. Accordingly, it is within the scope of this invention to further use a compound as described herein in the preparation (manufacture or formulation) of a composition such as a medicament, pharmaceutical composition, drug or vaccine compositions comprising the compound and a physiologically or pharmaceutically acceptable carrier, excipient or vehicle, and optionally other ingredients. Pharmaceutical compositions can be used as an anti-parasitic agent to treat malaria. Vaccine compositions can be used as an immunization agent to protect a subject from malaria.

In a still further aspect, the present invention relates to a computer-based method for modeling the three-dimensional structure of a target protein, comprising:
(a) obtaining a three-dimensional representation of an EBA-175 RII protein or a functional domain thereof;
(b) determining similarity between amino acid sequence of the target protein and that of the EBA-175 RII protein or the functional domain thereof; and
(c) constructing a three-dimensional structural model for the target protein based on the three-dimensional representation of an EBA-175 RII protein or the functional domain thereof.

Preferably, in the event that the amino acid sequence similarity between the target protein and the EBA-175 RII protein or the functional domain thereof is above a threshold level (e.g., above 30%, or more preferably, above 40%), the three-dimensional structural model for the target protein can be constructed by using a comparative modeling process. Alternatively, in the event that the amino acid sequence similarity between the target protein and the EBA-175 RII protein or the functional domain thereof is below a threshold level (e.g., below 40%, or more preferably, below 30%), the three-dimensional structural model for the target protein can be constructed by using a fold recognition or threading process.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts DBL containing proteins from Plasmodium species. FIG. 1A shows the ebl family shares a conserved gene structure, with 1 or 2 DBL domains in region II. The var superfamily contains multiple repeats of DBL domains. MAEBL is a unique member of the ebl family. It has the conserved gene structure but its region II does not contain DBL domains. Instead, the MAEBL ligand domains are related to another merozoite protein, apical membrane antigen 1 (AMA-1). PvDBP—Plasmodium vivax Duffy Binding Protein; PkDBP—Plasmodium knowlesi DBP; PfEMP-1—Plasmodium falciparum Erythrocyte Membrane Protein 1. FIG. 1B shows alignment and structural depiction of DBL containing proteins. Light blue indicates conserved residues, and deep blue indicates invariant residues. Cysteines are shown in red. Secondary structure is shown above the sequence alignment with F1 in green, linker regions in gold and F2 in purple. Dashed lines indicate regions not observed in the structure of RII. Blue and red dots indicate residues involved in dimerization and glycan binding, respectively. Sequences are from P. falciparum: EBA-175 (SEQ ID NO: 1), BAEBL (SEQ ID NO: 2), EBL-1 (SEQ ID NO: 3), JESEBL (SEQ ID NO: 4) and PEBL (SEQ ID NO: 5).

FIG. 2 depicts the RII monomer. FIG. 2A shows overall structure of the RII monomer. F1—green, F2—purple, linker—gold, bound sulfates—CPK spheres. FIG. 2B shows overlay of RII subdomains F 1 (green) and F2 (purple). Red bonds and yellow bonds indicate disulfide bridges in F1 and F2, respectively. FIG. 2C shows topology diagram of RII denoting similarity between F1 (green) and F2 (purple). Disulfide bridges (red) are all within sub-domains of F1 and F2. The dotted line indicates portions of the structure ordered in the complex but not in unbound RII.

FIG. 3 depicts the RII dimer. FIG. 3A shows stereo ribbon representation of the RII dimer. Monomers are shaded in different intensities (light/dark) for clarity. F1 domains are green or light green; F2 domains are purple or light purple, and the linker regions are gold or light gold. Sulfates are depicted in ball and stick (sulfurs in yellow, oxygens in red). FIG. 3B shows two views of the electrostatic surface potential of the RII dimer calculated by GRASP at an ionic strength of 0.1M. Surface potential is colored is from −10 kT (red) to +10 kT (blue). Sulfates can be clearly seen on the “bottom” surface on the left and in the channels. FIG. 3C shows a close up view of the b-finger of F1 (green) inserted into a cavity of F2 (purple). FIG. 3D shows a close up view of the b-finger of F2 (purple) and the cavity of F1 (green). FIG. 3E shows interactions between the two-stranded anti-parallel b-sheet of the two F2s. Coloring scheme is the same as in FIG. 3A. Dashed lines indicate hydrogen bonds and/or electrostatic interactions.

FIG. 4 depicts crystal structure of RII with sialyllactose. FIG. 4A shows ribbon diagram of RII with the position of the glycans indicated by the F_{obs}-F_{calc} electron density map contoured at 2.5 s in red. FIGS. 4B, 4C and 4D show close up view of three of the glycan binding sites 1, 3 and 5, respectively. All glycans are contacted by residues from both monomers. Color scheme is the same as in FIG. 3.

FIG. 5 depicts model for P. falciparum EBA-175-RII binding to the red blood cell receptor glycophorin A (GpA). The P. falciparum membrane is shown on the top and the erythrocyte membrane on the bottom. The receptor binding domain of EBA-175, RII, is shown as a surface representation with F1 in green and F2 in purple and the linker in gray. Blue lines represent portions of EBA-175 backbone not included in the crystal structure. GpA is shown in red with the membrane-spanning region in detail using the NMR structure and the extracellular domain drawn as a schematic flexible line. The modeled O-glycans of GpA (see FIGS. 7A-7D) are shown as space filling models in gold. In the left panel, the RII dimer assembles around the GpA dimer, with GpA binding within the channels (see text). An alternative model is shown on the right, where the GpA monomers dock on the outer surface of the protein, feeding glycans into the channels.

FIG. 6 depicts stereo representations of a possible model for the six glycans superimposed on FIG. 6A. Unbiased F_{obs}-F_{calc} electron density maps calculated prior to incorporating the glycans into the model. Red and blue—maps contoured at 3 and 2 σ respectively. FIG. 6B shows 2 F_{obs}-F_{calc} electron density map following refinement including the glycans contoured at 1 σ. FIG. 6C shows simulated-annealed F_{obs}-F_{calc} omit map contoured at 4 σ. FIG. 6D shows ribbon diagram of RII with the glycans shown in stick. FIGS. 6E-6G show close up view of three of the glycan binding sites, glycan 1, 3 and 5, respectively. Neu5Ac denotes sialic acid. H-bonds are drawn as dashed lines.

FIG. 7 depicts modeling of the complete GpA glycan. FIG. 7A illustrates the Siaα-2,3-Galβ-1,3 (Siaα2,6)GalNAc glycans, which are shown in stick. Glycans 1 and 2 are in yellow, glycans 3 and 4 in pink and glycans 5 and 6 are in cyan. The left panel shows a ribbon diagram and the right panel shows a surface representation with F1 in green, F2 in purple and the linker in gray. FIGS. 7B and 7C are the same as FIG. 7A, except rotated by 90° and 180°, respectively, along the x-axis. FIG. 7D is the same as FIG. 7C except rotated by 60° along the y-axis. Glycans 1/2 and 3/4 are found in the channels on the top and bottom surfaces of RII respectively. Glycans 5/6 are bound in a pocket formed between the two monomers, and are accessible by a small channel as seen in D right panel. Modeling indicates that all six full O-glycans can be easily accommodated at the observed binding sites in the complex structure, and that all the O-glycans are accessible from the surface of RII. FIG. 7E. shows a close-up view of glycan 1. FIG. 7F shows a close up view of glycan 3. FIGS. 7G and 7H show two close up views of glycan 5. Glycans 2,4 and 6 are equivalent to 1, 3 and 5, respectively. Coordinates for the O-glycan, obtained from PDB entry 2CWG (Wright and Jaeger, 1993), were used to create CNS topology files using the PRODRG2 server (Schuettelkopf and van Aalten, 2004). For glycans 1 and 3, each O-glycan was modeled into the structure by overlaying the atoms of the rings of the sialic acid group of Neu5Ac(α2-3)Gal on the sialic acid placed in the electron density. For glycans 5, the O-glycan was modeled by overlaying the atoms of the sialic acid and galactose groups. Cartesian molecular dynamics was performed in CNS, using atoms of the O-glycans and the protein (i.e. lacking water and sulfates). For glycans 1 and 3, the six atoms of the sialic acid ring were fixed but the remaining atoms of the O-glycan and a further 4 Å sphere of protein atoms around the glycan were free to move. For glycan 5, the atoms of the sialic acid and galactose rings were fixed. Following dynamics, minimization was performed in CNS, with the entire O-glycan and a 4 Å sphere of protein atoms around the glycan being allowed to move. Glycans 2, 4 and 6 were obtained from glycans 1, 3 and 5 respectively by applying matrices that related the two pairs of glycans obtained from the structure of the complex. Modeling was also done for the six glycans separately and minimization was carried out using NCS restraints resulting in a very similar model. Glycan geometry is well within reasonable values with RMSDs for bond lengths, angles and dihedrals for the glycans of 0.002 Å, 1.85° and 16.2°, respectively, with no violations.

FIG. 8 depicts functional analysis. FIG. 8A illustrates a rosette (red blood cells adherent to a COS-7 cell expressing RII on its surface) which is shown on the left. Unbound COS-7 cells are shown on the right; FIG. 8B shows immunofluorescence images of cell surface expression of RII and mutants. Left panel—nuclei staining with Hoechst 33342 (blue), Middle panel—immunofluorescence of surface expressed RII using a FITC conjugated secondary antibody (green), Right panel—merge of left and middle panel. All images were recorded at the same magnification with the scale bar in top left panel corresponding to 50 μm.

FIG. 9 depicts mapping of inhibitory peptides onto the structure of RII. Peptides 355-375 and 435-455 are shown in orange and red respectively. The remaining portions of RII are shown in gray. The glycans are shown in ball and stick.

### DETAILED DESCRIPTION OF THE INVENTION

The present invention provides crystals of a Duffy binding like (DBL) domain of erythrocyte-binding like (ebl) proteins, particularly, erythrocyte binding antigen 175 region II (EBA-175 RII). In a particular embodiment, crystals of EBA-175 RII have the three-dimensional structure defined by the atomic coordinates of Table 5 or 7. The atomic coordinates of Table 5 or Table 7 provide a measure of atomic location in Angstroms (A). The coordinates are a relative set of positions that define a shape in three dimensions, but the skilled person would understand that an entirely different set of coordinates having a different origin and/or axes could define a similar or identical shape.

By “root mean square deviation” is meant the square root of the arithmetic mean of the squares of the deviations from the mean. One skilled in the art would understand that varying the relative atomic positions of the atoms of the structure so that the root mean square deviation of the residue backbone atoms (i.e. the nitrogen-carbon-carbon backbone atoms of the protein amino acid residues) of each domain of a DBL domain protein, e.g., F1 or F2 of EBA-175 RII, is less than 2.5 A (preferably less than 1.0 A and more preferably less than 0.5 A) when superimposed on the coordinates provided in Table 5 or 7 for the residue backbone atoms, will generally result in a structure which is substantially the same as the structure of Table 5 or 7 in terms of both its structural characteristics and potency for structure-based design of ebl family protein inhibitors, e.g., EBA-175 inhibitors. Similarly, the skilled person would understand that changing the number and/or positions of the water molecules and/or substrate molecules of Table 5 or 7 will not generally affect the potency of the structure for structure-based design of EBA-175 inhibitors. Thus for the purposes described herein as being aspects of the present invention, it is within the scope of the invention if the Table 5 or 7 coordinates are transposed to a different origin and/or axes; the relative atomic positions of the atoms of the structure are varied so that the root mean square deviation of residue backbone atoms of each domain of a DBL domain protein, e.g., F1 or F2 domain of EBA-175 RII, is less than 2.5 A (preferably less than 1.0 A and more preferably less than 0.5 A) when superimposed on the coordinates provided in Table 5 or 7 for the residue backbone atoms; and/or the number and/or positions of water molecules and/or substrate molecules is varied. Reference herein to the coordinate data of Table 5 or 7 thus includes the coordinate data in which one or more individual values of Table 5 or 7 are varied in this way.

Thus, for example, varying the atomic positions of the atoms of the structure by up to about 5 A, preferably up to about 2.5 A, and more preferably up to about 1 A, in any direction will result in a structure which is substantially the same as the structure of Table 5 or 7 in terms of both its structural characteristics and utility e.g. for structure-based drug design.

The present invention identifies atomic coordinates of at least one receptor binding site on a DBL domain, particularly, EBA-175 RII, that specifically binds to its erythrocytic ligand or receptor glycophorin A (GpA). In one embodiment, the present invention provides atomic coordinates of six GpA-receptor-binding sites on EBA-175 RII (i.e., EBA-175 erythrocyte-binding domain). In a particular embodiment, the present invention identifies six receptor bind sites comprising residues N417, R422, N429, K439 and D442 of one monomer of a RII dimer and K28 of the other monomer (receptor binding sites 1 and 2), residues N550, N551, Y552, K553 and M554 from one monomer of the RII dimer and N33 from the other monomer (receptor binding sites 3 and 4, residues T340, K341, D342, V343, Y415, Q542 and Y546 of one monomer of the RII dimer and residues K28, N29, R31 and S32 of the second monomer (receptor binding sites 5 and 6).

In another embodiment, three-dimensional structural information is obtained from a crystal of EBA-175 RII (form 1) having unit cell dimensions of about a=76.96 A, b=76.96 A, c=277.33 A and alpha=beta=gamma=90 and/or a crystal of EBA-175 RII (form 2) having unit cell dimensions of about a=103.65 A, b=103.65 A, c=212.72 A and alpha=beta=gamma=90. In a particular embodiment, the crystals of form 1 form in space group P4₃2₁2 and the crystals of form 2 form in space group space group P4₁22. The present invention also contemplates crystals which form in other space groups.

In still another embodiment, three-dimensional structural information of RII is obtained from a crystal of EBA-175 RII-ligand complex, e.g., EBA-175 RII-sialy lactose complex (form 3), having unit cell dimensions of about a=145.74 A, b=146.21 A, c=214.74 A and alpha=beta=gamma=90. The crystal of form 3 has a space group of C222₁.

Modifications in the EBA-175 RII crystal structure due to, for example, mutations, additions, substitutions, and/or deletions of amino acid residues (including the deletion of one or more EBA-175 RII promoters) (“variants”) could account for variations in the EBA-175 RII atomic coordinates. Atomic coordinate data of EBA-175 RII modified so that the receptor that bound to one or more binding sites of EBA-175 RII would be expected to bind to the corresponding binding sites of the modified EBA-175 RII are encompassed by the present invention. Reference herein to the coordinates of Table 5 thus includes the coordinates modified in this way. Preferably, the modified coordinate data define at least one EBA-175 RII binding cavity.

Examination of the EBA-175 RII crystal structure reveals that the elongated RII molecule forms a dimer, interacting extensively along the elongated surface in an antiparallel fashion. The dimeric organization displays two prominent channels at the center of dimer. The two domains, F1 and F2, are mostly helical and similar in structure. Of the 27 cysteine residues within RII, 26 are involved in disulfide bonds while the remaining free thiol is exposed on the surface. Several sulfates are identified in the crystal structure and are bound to the channels and on one surface.

Any crystallization technique known to those skilled in the art may be employed to obtain the crystals of the invention, including, but not limited to, vapor diffusion (either by sitting drop or hanging drop), batch crystallization or micro dialysis. In a particular embodiment, the crystals of the invention are obtained by vapor diffusion. Seeding of the crystals in some instances may be required to obtain X-ray quality crystals. Standard micro and/or macro seeding of crystals may therefore be used.

The crystals of the invention diffract to a resolution at least equal to or greater than (numerically less than) 2.3 A (2.25 A).

One aspect of the present invention provides a method for crystallizing the DBL domain protein, such as EBA-175 RII domain, for determining the three-dimensional atomic structure of the DBL domain protein, which comprises mixing a protein solution of the DBL domain protein a reservoir solution in any crystallization setup (preferably, but not exclusively, hanging drop, sitting drop, free liquid diffusion, batch or micro-batch), crystallizing the mixture in a closed container at a temperature of about 0° C. to about 37° C., preferably, 17° C., for a sufficient time, e.g., ranging from several hours to several weeks, preferably, 2 days, to form the crystals of the DBL domain protein, wherein the protein solution contains about 1 mg/ml to about 30 mg/ml DBL domain protein, e.g., EBA-175 RII protein, and in 1-100 mM buffer that can hold a pH value of about 5.5-9.0 and about 0-400 mM salt, e.g., NaCl or equivalent. More preferably, the protein solution contains EBA-175 RII at a concentration of about 12 or about 15 mg/ml, and further contains about 10 mM Tris-HCl and about 100 mM NaCl and has a pH value of about 7.4. The reservoir solution contains either about 0.1-0.4 M ammonium sulfate, about 0.01-0.2 M buffer that can hold a pH value of about 5.5-9.0, and about 10-35% polyethylene glycol or polyethylene glycol monomethyl ether having a molecular weight (MW) of about 1,000 to about 20,0000, or about 1.5-3.5 M ammonium sulfate, about 0.01-0.2 M buffer that can hold a pH value of about 5.5-9.0, and about 0.01-10% polyethylene glycol or polyethylene glycol monomethyl ether having a molecular weight (MW) of about 100 to about 1,000. Preferably, the reservoir solution contains either about 0.265 M ammonium sulfate, about 0.1 M sodium cacodylate at pH value of about 6.5 and about 29% polyethylene glycol 8000 for a protein solution of about 15 mg/ml or about 2.6-2.8 M ammonium sulfate, about 0.1 M sodium cacodylate at pH value of about 6.5 and about 0.05-2% polyethylene glycol 750 monomethyl ether for a protein solution of about 12 mg/ml.

Another aspect of the present invention provides a method for co-crystallizing a DBL domain protein/receptor or ligand complex, e.g., the EBA-175 RII/receptor or ligand complex, for determining the receptor binding sites on three-dimensional atomic structure of EBA-175 RII, which comprises mixing a protein solution of DBL domain, e.g., EBA-175 RII, with a reservoir solution, and a ligand solution in any crystallization setup (preferably, but not exclusively, hanging drop, sitting drop, free liquid diffusion, batch or micro-batch), crystallizing the mixture in a closed container at a temperature of about 0° C. to about 37° C., preferably, 17° C., for a sufficient time, e.g., for a period of time ranging from several hours to several weeks, preferably, 2 days, to form the crystals of the DBL domain protein, wherein the protein solution contains about 1 mg/ml to about 30 mg/ml DBL domain protein, e.g., EBA-175 RII protein, and in 1-100 mM buffer that can hold pH about 5.5-9.0 and about 0-400 mM salt, e.g., NaCl or equivalent. More preferably, the protein solution contains EBA-175 RII at a concentration of about 12 or about 15 mg/ml, and further contains about 10 mM Tris-HCl and about 100 mM NaCl and has a pH value of about 7.4. The reservoir solution contains either about 0.1-0.4 M ammonium sulfate, about 0.01-0.2 M buffer that can hold pH about 5.5.-9.0, and about 10-35% polyethylene glycol or polyethylene glycol monomethyl ether having a molecular weight (MW) of about 1,000 to about 20,0000, or about 1.5-3.5 M ammonium sulfate, about 0.01-0.2 M buffer that can hold pH about 5.5.-9.0, and about 0.01-10% polyethylene glycol or polyethylene glycol monomethyl ether having a molecular weight (MW) of about 100 to about 1,000. Preferably, the reservoir solution contains either about 0.265 M ammonium sulfate, about 0.1 M sodium cacodylate at pH about 6.5 and about 29% polyethylene glycol 8000 for a protein solution of about 15 mg/ml or about 2.6-2.8 M ammonium sulfate, about 0.1 M sodium cacodylate at pH about 6.5 and about 0.05-2% polyethylene glycol 750 monomethyl ether for a protein solution of about 12 mg/ml. The ligand solutionused above, preferably, an α-2,3-sialyllactose solution, can have a concentration of about 1 nM-100 mM, preferably, about 10 mM.

In a particular embodiment, the compound that mimics a GpA receptor and contains the essential components required for binding (i.e., sialic acid) is Neu5Ac(α2-3)Gal or α-2,3-sialyllactose.

According to the present invention, buffers that can hold pH 5.5-9.0 include, but are not limited to, Tris, Imidazole, sodium cacodylate, MES, HEPES, MOPS.

Any crystallization technique known to those skilled in the art can be employed to obtain the crystals of the present invention, including, but not limited to, vapor diffusion (either by sitting drop or hanging drop), batch crystallization and micro dialysis. In a particular embodiment, the crystallization occurs by using hanging drops and vapor diffusion method to a resolution of less than about 2.3 A. In another particular embodiment, derivatized crystals of RII can be obtained by soaking RII crystals in an appropriate solution, preferably, saturated lithium sulfate containing either sodium dicyanoaureate or potassium tetrachloroplatinate for a sufficient period of time, preferably, 2 hours.

The three-dimensional structural information obtained from the crystals of the present invention provides a means for designing new candidate compounds which have the potential to inhibit binding of EBA-175 to glycophorin A, to erythrocytes or to both glycophorin A and erythrocytes, thereby halting and/or blocking merozite invasion into erythrocytes. The present invention also contemplates other uses of atomic structural information of EBA-175 RII identified by the present invention, including, but not limited to, protein structure prediction, vaccine design or structure-assisted design. The phrase “structure-assisted design” as used herein refers to a method in which a pre-designed or pre-selected candidate ligand or drug is modified or optimized based on the three-dimensional structural information of the target molecule, e.g., an enzyme or receptor protein molecule. The structure-assisted design thus is different from, and complements, the de novo drug design.

The determination of the three-dimensional structure of EBA-175 RII provides a basis for design of new and specific modulator compounds for EBA-175 RII. For example, computer-modeling programs can be used to design different compounds expected to bind or interact with confirmed or putative binding sites or other structural or functional features of EBA-175 RII employing the three-dimensional structure of EBA-175 RII.

A “modulator compound” is defined herein to include inhibitors of EBA-175 and compounds which affect its specificity or activity in other ways. An “inhibitor” is an agent that acts by inhibiting (preventing or decreasing) at least one function characteristic of the interaction between EBA-175 RII and erythrocytes and/or glycophorin A, such as a binding activity (complex formation), cellular signaling triggered by the interaction and/or cellular response function (e.g., pathogenicity) mediated by the interaction. An inhibitor includes agents that bind either EBA-175 RII or an EBA-175 RII receptor (glycophorin A, erythrocytes, sialic acid) (e.g., an antibody, a mutant of a natural ligand, a peptidomimetic, and other competitive inhibitors of ligand binding), and to substances that inhibit a function mediated by complex formation between EBA-175 RII and its receptor without binding thereto (e.g., an anti-idiotypic antibody). A “candidate modulator compound” is a molecule, be it naturally-occurring or artificially-derived, and includes, for example, peptides, proteins, including antibodies and antibody fragments, peptidomimetics, synthetic molecules, for example, synthetic organic molecules, ionic compounds, naturally-occurring molecules, for example, naturally occurring organic molecules, nucleic acid molecules, and components thereof.

The step of providing a structure of a candidate compound may involve selecting the compound by computationally screening a database of compounds for binding or interaction with EBA-175 RII. For example, a three-dimensional descriptor for the candidate compound may be derived, the descriptor including geometric and functional constraints derived from the architecture and chemical nature of the binding site or interacting surface. The descriptor may then be used to interrogate the compound database, with a candidate compound being a compound that has a good match to the features of the descriptor. In effect, the descriptor is a type of virtual pharmacophore.

By “binding site” or “binding pocket” is meant a (typically three-dimensional) portion of EBA-175 RII containing one or more receptor interaction sites. By “interaction site” is meant a site (such as an atom, a functional group of an amino acid residue or a plurality of such atoms and/or groups) in a EBA-175 RII binding pocket which may bind to a candidate or potential inhibitor compound. Sites may exhibit attractive or repulsive binding interactions due to charge, steric considerations and the like depending on the molecule in the cavity.

Particular binding sites of EBA-175 RII include those identified as confirmed or putative binding sites/regions based on the atomic coordinates of Table 5 (e.g., surface of a channel surface created by a dimer of EBA-175 RII, binding site of surface sulfates, sialic binding sites, binding site of EBA-175 region II that binds an antibody or antibody fragment).

The present invention permits the use of molecular design techniques to design, identify and synthesize candidate compounds capable of binding or interacting with EBA175 RII. The atomic coordinates of EBA-175 RII may be used in conjunction with computer modeling using a docking program such as eHiTS, GRAM, DOCK, HOOK or AUTODOCK (Dunbrack et al., Foldings & Design, 2:27-42 (1997)) to identify candidate compounds of EBA-175 RII. This procedure can include computer fitting of candidate compounds to binding sites of EBA-175 RII to ascertain how well the shape and structure of the candidate compound will complement the binding sites or to compare the candidate compounds with the binding of sialic acid, for example, in the binding sites (West, M. L. et al., Trends Pharmacol. Sci., 16:67-74 (1995)). Computer programs may also be employed to estimate the attraction, repulsion and steric hindrance of the EBA-175 RII and candidate compound. Generally, the tighter the fit, the lower the steric hindrances, the greater the attractive forces and the greater the specificity which are important features for a specific compound which is more likely to interact with EBA-175 RII rather than other classes of proteins.

By “fitting” is meant determining by automatic or semi-automatic means, interactions between one or more atoms of a candidate modulator compound and one or more atoms of EBA-175 RII, and calculating the extent to which such interactions are stable. Interactions include attraction and repulsion brought about by charge, steric considerations and the like. Various computer-based methods for fitting are described further herein.

The present invention also permits the use of molecular design techniques to computationally screen databases for compounds that can bind to EBA-175 RII in a manner analogous to sialic acid, for example. Molecular databases that can be screened include, but are not limited to, Protein Data Bank (PDB) (Brookhaven National Laboratory, Upton, N.Y.), Cambridge Structural Database (CSD) (Crystallographic Data Centre, University Chemical Laboratory, Cambridge, UK), Available Chemical Directory (ACD) (MDL Information Systems, Inc., San Leandro, Calif.), Triad and Iliad (University of California, Berkeley, Calif.), Chemical Abstracts Service (Columbus, Ohio), etc.

The compounds of the present invention may also be designed by visually inspecting the three-dimensional structure to determine more effective modulators of EBA-175 RII. This type of modeling may be referred to as manual drug design. Manual drug design may employ visual inspection and analysis using graphics visualization program such as Insight, Sybyl, and 0 (Jones, T. A. et al., Acta Crystallog., 47 (Pt 2):110-119 (1991)).

The compounds designed using the information of the invention may be competitive or noncompetitive molecules. Designed compounds which have favorable (desirable) properties (e.g., strong attraction between the candidate designed compound and EBA-175 RII), can be screened for activity to determine the ability of the compound to interact or bind with EBA-175 RII. Adjustments can be made to the structure or functionality of the candidate compound (e.g., to improve binding to the binding site), if appropriate, based on the detailed structural information. These steps can be repeated as needed. See, e.g., Sternberg, M. J. E. (ed.), Protein Structure Prediction: A Practical Approach (Oxford, UK: Oxford University Press) (1996), which teachings are entirely incorporated herein by reference. These designed compounds may bind to all or a portion of a binding site of EBA-175 and may be more potent, more specific, less toxic and more effective than known binding entities for EBA-175. The designed compounds may also be less potent but have a longer half life in vivo and/or in vitro and therefore be more effective at inhibiting interaction of EBA-175 with glycophorin A, erythrocytes or both glycophorin A and erythrocytes in vivo and/or in vitro for prolonged periods of time. Designed compounds that bind to EBA-175 can be screened for activity (e.g., the ability to inhibit binding of EBA-175 to glycophorin A, to erythrocytes or to both glycophorin A and erythrocytes and the ability to block or inhibit merozite invasion of erythrocytes).

The three-dimensional structural information and atomic coordinates associated with the structural information of EBA-175 RII bound to an inhibitory compound is useful in a rational drug design providing for a method of identifying candidate compounds which bind to or interact with EBA-175 RII. The method for identifying said potential inhibitor for EBA-175 RII comprises (a) using a three-dimensional structure of EBA-175 RII as defined by its atomic coordinates listed in Table 5; (b) employing said three-dimensional structure to design or select said candidate compound; (c) synthesizing said candidate compound; (d) determining whether said compound is capable of forming a complex with EBA-175 region II or a functional variant thereof.

Those skilled in the field of drug discovery and development will understand that the precise source of compounds is not critical to the three-dimensional computational screening methods of the invention. Accordingly, virtually any number of compounds can be designed using the information described herein. Compounds designed using the information herein may be those found in extracts including, but not limited to, plant-, fungal-, prokaryotic- or animal-based extracts, fermentation broths. The compounds may be synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic small molecule libraries are commercially available, e.g., from Brandon Associates (Merrimack, N.H.) and Aldrich Chemical (Milwaukee, Wis.). In another embodiment, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.).

In addition, natural and synthetically produced molecules or libraries can be generated by any suitable method (e.g., by standard extraction and fractionation methods). For example, candidate compounds can be obtained using any of the numerous approaches in combinatorial library methods, including biological libraries; spatially addressable parallel solid phase or solution phase libraries; synthetic library methods requiring deconvolution; the “one-bead one-compound” library method; and

synthetic library methods using affinity chromatography selection. The biological library approach is limited to polypeptide libraries, while the other four approaches are applicable to polypeptide, non-peptide oligomer or small molecule libraries of compounds (Lam, Anticancer Drug Des., 12:145, 1997). Furthermore, if desired, any library or compound can be readily modified using standard chemical, physical, or biochemical methods.

The term “antibody” as used herein refers to immunoglobulin molecules and immunologically active portions of immunoglobulin molecules, i.e., molecules that contain an antigen-binding site that selectively binds an antigen (e.g., an antigen-binding fragment of an antibody). Antibodies can be polyclonal or monoclonal, and the term antibody is intended to encompass both polyclonal and monoclonal antibodies. The terms polyclonal and monoclonal refer to the degree of homogeneity of an antibody preparation, and are not intended to be limited to particular methods of production.

Antibodies designed or selected using the information herein can be raised against an appropriate immunogen, including proteins or polypeptides of the EBA-175 (including synthetic molecules, such as synthetic peptides).

Preparation of immunizing antigen, and polyclonal and monoclonal antibody production can be performed using any suitable technique. A variety of methods have been described (see e.g., Kohler et al., Nature, 256:495-497, 1975 and Eur. J. Immunol., 6:511-519, 1976; Milstein et al., Nature, 266:550-552, 1977; Koprowski et al., U.S. Pat. No. 4,172,124; Harlow, E. and D. Lane, Antibodies: A Laboratory Manual, (Cold Spring Harbor Laboratory: Cold Spring Harbor, N.Y.) (1988); and Current Protocols In Molecular Biology, Vol. 2 (Supplement 27, Summer '94), Ausubel, F. M. et al., Eds., (John Wiley & Sons: New York, N.Y., Chapter 11 (1991); the teachings of each of which are incorporated herein by reference). Generally, a hybridoma is produced by fusing a suitable immortal cell line (e.g., a myeloma cell line such as SP2/0) with antibody producing cells. The antibody producing cell, preferably those of the spleen or lymph nodes, can be obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Other suitable methods of producing or isolating antibodies of the requisite specificity can be used, including, for example, methods which select recombinant antibody from a library, or which rely upon immunization of transgenic animals (e.g., mice) capable of producing a full repertoire of human antibodies (see e.g., Jakobovits et al., Proc. Natl. Acad. Sci. USA, 90:2551-2555, 1993; Jakobovits et al., Nature, 362:255-258, 1993; Lonberg et al., U.S. Pat. No. 5,545,806; and Surani et al., U.S. Pat. No. 5,545,807; the teachings of which are each incorporated herein by reference).

Single-chain antibodies, and chimeric, humanized or primatized (CDR-grafted), or veneered antibodies, as well as chimeric, CDR-grafted or veneered single-chain antibodies, comprising portions derived from different species, and the like are also encompassed by the present invention and the term “antibody.” The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Patent No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460, 1992, regarding primatized antibody, and Ladner et al., U.S. Pat. No. 4,946,778 and Bird, R. E. et al., Science, 242:423-426, 1988 regarding single-chain antibodies.

In addition, antigen-binding fragments of antibodies, including fragments of chimeric, humanized, primatized, veneered or single-chain antibodies, can also be produced. Antigen-binding fragments of foregoing antibodies retain at least one binding function and/or modulation function of the full-length antibody from which they are derived. For example, antigen-binding fragments, include, but not limited to, Fv, Fab, Fab′ and F(ab′)₂ fragments. Such fragments can be produced by enzymatic cleavage or by recombinant techniques. For instance, papain or pepsin cleavage can generate Fab or F(ab′)₂ fragments, respectively. Reduction of the disulfide bond between the heavy chains of F(ab′)₂ fragments can yield F(ab′) fragments. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH, domain and hinge region of the heavy chain.

Anti-idiotypic antibodies recognize antigenic determinants associated with the antigen-binding site of another antibody. Anti-idiotypic antibodies can be prepared against a first antibody by immunizing an animal of the same species, and preferably of the same strain as the animal used to produce the first antibody, with said first antibody. See e.g., U.S. Pat. No. 4,699,880.

The three-dimensional structural information and atomic coordinates associated with the structural information of EBA-175 RII are useful for solving the structure of crystallized proteins which are thought to be similar in structure based on function or sequence similarity or identity to EBA-175 RII. For example, the structure of crystallized proteins thought to be similar to EBA-175 RII can be determined by molecular replacement, multiple isomorphous replacement (MIR) analysis, multiple anomaly dispersion (MAD), etc. Examples of computer programs known in the art for performing molecular replacement are CNS (Brunger et al., Current Opin. Struct. Biol., 8 (5):606-611 (1998)); commercially available from Accelerys, San Diego, Calif.) and AMORE (Navaza Acta Crystallographica, A50:157-163 (1994)) and Phaser (Storoni et al., Acta Crystallogr D Biol Crystallogr 60, 432-38 (2004)).

Molecular replacement refers to a method that involves generating a preliminary model of the three-dimensional structure of a crystallized protein of the invention whose structure coordinates are unknown prior to employment of molecular replacement. Molecular replacement is achieved by orienting and positioning a molecule whose structure coordinates are known (in this case the EBA-175 RII having the atomic coordinates of Table 5) within the unit cell as defined by X-ray diffraction pattern obtained from a crystallized protein whose structure is unknown so as to best account for the observed diffraction pattern of the unknown crystal. Phases can then be calculated from this model and combined with the observed amplitudes to give an approximate Fourier synthesis of the structure whose coordinates are unknown. This in turn can be subject to any of several forms of refinement to provide a final, accurate structure.

The present invention also provides systems, particularly computer systems, intended to generate structures and/or perform rational drug design for EBA-175 RII or a complex of EBA-175 RII and candidate inhibitor compound, wherein the systems comprise either (a) atomic coordinate data according to Table 5, said data defining the three-dimensional structure of EBA-175 RII or (b) structure factor data derived from the atomic coordinate data of Table 5.

The invention further provides computer readable media with either (a) atomic coordinate data according to Table 5, said data defining the three-dimensional structure of EBA-175 RII or (b) structure factor data recorded thereon, wherein the structure factor data are derived or calculated from the atomic coordinate data of Table 5.

By “computer readable media” is meant any media which can be read and accessed directly by a computer. Such media include, but are not limited to, magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By providing computer readable media, the atomic coordinate data can be routinely accessed to model EBA-175 RII or a subdomain thereof. For example, RASMOL (Sayle et al., Trends Biochem. Sci., 20 (9):374 (1995)) is a publicly available computer software package which allows access and analysis of atomic coordinate data for structure determination and/or rational drug design.

Structure factor data, which are derived from atomic coordinate data (see, e.g., Blundell et al., in Protein Crystallography (New York: Academic Press) (1976)), are useful for calculating, e.g., Fourier electron density maps.

By “computer system” is meant a hardware means, software means and data storage means used to analyze the atomic coordinate data of the invention. The minimum hardware means of the computer-based systems of the invention comprises a central processing unit (CPU), input means, output means and data storage means. Preferably, a monitor is provided to visualize structure data. The data storage means may be RAM or means for accessing computer readable media of the invention. Examples of such systems are microcomputer workstations available from Silicon Graphics, Inc., Sun Microsystems running Unix based Windows NT or IBM OS/2 operating systems, PC systems, Macintosh systems, and the like.

The present invention also pertains to the crystal structure of pharmaceutical compositions comprising compounds that interact or bind to EBA-175 RII, identified as described herein. The compounds can be formulated with a physiologically or pharmaceutically acceptable carrier or excipient to prepare a pharmaceutical composition. The carrier and composition can be sterile. The formulation should suit the mode of administration.

Suitable physiologically and pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The pharmaceutical composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy, rechargeable or biodegradable devices, particle acceleration devises (“gene guns”) and slow release polymeric devices. The pharmaceutical compositions of this invention can also be administered as part of a combinatorial therapy with other compounds.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active compound. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., that are, if desired, sterilized or mixed with auxiliary agents, e.g., preservatives, stabilizers, wetting agents, buffers or salts for influencing osmotic pressure, etc. The compound may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Therapeutics described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, that notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the compounds can be separated, mixed together in any combination, present in a single vial or tablet. Compositions assembled in a blister pack or other dispensing means is preferred. For the purpose of this invention, unit dosage is intended to mean a dosage that is dependent on the individual pharmacodynamics of each compound and administered in FDA approved dosages in standard time courses.

Pharmaceutical compositions of the present invention can be used as an anti-parasitic agent to treat malaria.

The importance of RII as a vaccine candidate has been established. In vaccine studies, RII was highly immunogenic, and protected 3 of 4 Aotus monkeys immunized with a DNA plasmid prime and recombinant protein boost regimen from P. falciparum challenge (Sim, B. K. et al., Mol. Med., 7:247-254 (2001); Jones, T. R. et al., J. Infect. Dis., 183:303-312 (2001)). In addition, RII is highly conserved among more than 30 laboratory and field isolates studied (Liang, H. and Sim, B. K., Mol. Biochem. Parasitol., 84:241-245 (1997)).

The present invention also provides a method for the rational design of vaccine compositions comprising (a) providing a three-dimensional structure of EBA-175 RII; (b) determining one or more receptor binding site or oligomerization interface from said three-dimensional structure of EBA-175 RII; (c) providing an amino acid sequence of a EBA-175 RII peptide which includes one or more said receptor binding site and/or oligomerization interface; and (d) formulating said peptide of step (c) as a vaccine composition. Receptor binding sites of EBA-175 RII include those identified as confirmed or putative binding sites/regions based on the atomic coordinates of Table 5 (e.g., surface of a channel surface created by a dimer of EBA-175 RII, binding site of surface sulfates, sialic binding sites, binding site of EBA-175 RII that binds an antibody or antibody fragment). By “oligomerization interface” is meant a dimerization interaction site or dimerization interface (such as an atom, a functional group of an amino acid residue or a plurality of such atoms and/or groups) of EBA-175 RII that mediates interactions between EBA-175 RII molecules. Oligomerization or dimerization interaction sites of EBA-175 RII include those identified as confirmed or putative interaction sites/regions based on the atomic coordinates of Table 5 (e.g., sites of EBA-175 RII that mediate interactions between EBA-175 RII molecules).

Vaccine compositions can be formulated with a physiologically or pharmaceutically acceptable carrier or excipient. The carrier and composition can be sterile. The formulation should suit the mode of administration. Suitable physiologically and pharmaceutically acceptable carriers include those described herein for pharmaceutical compositions. Vaccine compositions can also be formulated as described herein for pharmaceutical compositions/therapeutics. Vaccine compositions can be administered as described for pharmaceutical compositions/therapeutics.

The present invention also features a method of producing a vaccine composition, comprising reducing the pathogenicity of a parasitic agent which comprises a gene encoding EBA-175 RII binding ligand, by producing a derivative of the parasitic agent which comprises a variant of the gene encoding a protein having reduced EBA-175 RII binding activity relative to that of EBA-175; and preparing a vaccine composition comprising said derivative. Such EBA-175 binding ligands that exhibit reduced EBA-175 RII binding capacity and/or reduced pathogenicity in vivo can be used in vaccine compositions. Such vaccines can be used therapeutically and/or for research purposes (e.g., to identify therapeutic agents that can reducing the pathogenicity of a parasitic agent).

Vaccine compositions of the present invention can also be used as an immunization agent to protect a subject from malaria.

The present invention also templates use of the three-dimensional structure of the EBA-175 RII protein to predict the unknown structure of a target protein.

Preferably, in the event that the amino acid sequence similarity between the target protein and the EBA-175 RII protein or the functional domain thereof is above a threshold level (e.g., above 30%, or more preferably, above 40%), the three-dimensional structural model for the target protein can be constructed by using a comparative modeling process. Alternatively, in the event that the amino acid sequence similarity between the target protein and the EBA-175 RII protein or the functional domain thereof is below a threshold level (e.g., below 40%, or more preferably, below 30%), the three-dimensional structural model for the target protein can be constructed by using a fold recognition or threading process.

The comparative modeling process, which is commonly used in the field of bioinformatics, predicts the unknown structure of a target protein based on the known structure of a template protein that is homologous (i.e., having high sequence similarity) to the target protein.

The fold recognition or threading technique, which is another commonly used protein structure prediction methodology in the field of bioinformatics, can be used to predict the 3D-structure of a target protein in the event that the target protein is not homologous (i.e., having low sequence similarity) to the template protein. Based on the fact that protein structures are typically conversed over sequence variations and that many non-homologous proteins have very similar structures or “folds,” the fold recognition or threading process predicts the unknown structure of the target protein by fitting the amino acid sequence of the target protein into the 3D-structure of the template protein (i.e., “threading” the target protein sequence through the 3-D structure of the template protein).

Both the comparative modeling process and the fold recognition or threading process require a protein database that contains multiple template proteins with known 3D-structures, and the presence of at least one template protein whose 3D-structure is substantially similar to that of the target protein in such a protein database is essential for successful prediction of the target protein structure using these two techniques.

Since the EBA-175 RII protein contains a novel structure or “fold,” the EBA-175 RII protein is useful in a protein database as a template protein, and the 3D-structure of the EBA-175 RII protein can be readily used to predict structures of target proteins that are homologous to the EBA-175 RII protein or have similar structures or folds to that of the EBA-175 RII protein.

The present invention will now be illustrated by the following examples, which are not to be considered limiting in any way.

### EXAMPLE 1

### Materials and Methods

The native RII domain of EBA-175 is unglycosylated in P. falciparum due to the lack of post-translational modification capabilities for glycosylation (Gowda and Davidson, 1999). However, expression in Pichia pastoris resulted in a glycosylated protein that was refractory to crystallization. A synthetic gene fragment encoding RII with 5 putative glycosylation sites mutated (N3Q, S50A, S 195A, T206A, N400Q) was cloned and expressed in Pichia pastoris, a methylotropic yeast. Fermentation was performed with defined salt media.

Purification was initiated by expanded bed adsorption chromatography, i.e., sulphopropyl cation exchange chromatography (Amersham Pharmacia Biotech). followed by additional ionic exchange chromatographic steps (SP chromatography Streamline SP XL resin, and Q FF (Pharmacia)). The final steps of the purification included a Streamline SP/XL cation exchange polishing step and an ultrafiltration diafiltration concentration step.

Plasmids Used in Construction of Pichia pastoris RII-N-gly- Production Clone:

3D7DEF-4: plasmid containing synthetic EBA-175 RII mammalian codon optimized gene from the 3D7 strain of P. falciparum with the five putative N-linked glycosylation sites conservatively mutated (N3Q, S50A, S195A, T206A, N400Q) (SEQ ID NO: 7) (gene identified as eba-175 RII-N-gly ⁻(SEQ ID NO: 6)). The synthetic EBA-175 RII mammalian codon optimized gene was subsequently mutated to remove the putative N-linked glycosylation sites (identified here as eba-175 RII-N-gly₋) by Retrogen, Inc. (San Diego, Calif.).

pPICZαA: P. pastoris expression vector (Invitrogen, Inc., San Diego, Calif.).

EBA-175 RII-N-gly DNA sequence (eba-175-N-gly):

(SEQ ID NO: 6)

GGCCGCCAGACCTCCTCCAACAACGAGGTGCTGTCCAACTGCCGCGAGAA
GCGCAAGGGCATGAAGTGGGACTGCAAGAAGAAGAACGACCGCTCCAACT
ACGTGTGCATCCCCGACCGCCGCATCCAGCTGTGCATCGTGAACCTGGCC
ATCATCAAGACCTACACCAAGGAGACCATGAAGGACCACTTCATCGAGGC
CTCCAAGAAGGAGTCCCAGCTGCTGCTGAAGAAGAACGACAACAAGTACA
ACTCCAAGTTCTGCAACGACCTGAAGAACTCCTTCCTGGACTACGGCCAC
CTGGCCATGGGCAACGACATGGACTTCGGCGGCTACTCCACCAAGGCCGA
GAACAAGATCCAGGAGGTGTTCAAGGGCGCCCACGGCGAGATCTCCGAGC
ACAAGATCAAGAACTTCCGCAAGAAGTGGTGGAACGAGTTCCGCGAGAAG
CTGTGGGAGGCCATGCTGTCCGAGCACAAGAACAACATCAACAACTGCAA
GAACATCCCCCAGGAGGAGCTGCAGATCACCCAGTGGATCAAGGAGTGGC
ACGGCGAGTTCCTGCTGGAGCGCGACAACCGCGCCAAGCTGCCCAAGTCC
AAGTGCAAGAACAACGCCCTGTACGAGGCCTGCGAGAAGGAGTGCATCGA
CCCCTGCATGAAGTACCGCGACTGGATCATCCGCTCCAAGTTCGAGTGGC
ACACCCTGTCCAAGGAGTACGAGACCCAGAAGGTGCCCAAGGAGAACGCC
GAGAACTACCTGATCAAGATCTCCGAGAACAAGAACGACGCCAAGGTGTC
CCTGCTGCTGAACAACTGCGACGCCGAGTACTCCAAGTACTGCGACTGCA
AGCACACCACCACCCTGGTGAAGTCCGTGCTGAACGGCAACGACAACACC
ATCAAGGAGAAGCGCGAGCACATCGACCTGGACGACTTCTCCAAGTTCGG
CTGCGACAAGAACTCCGTGGACACCAACACCAAGGTGTGGGAGTGCAAGA
AGCCCTACAAGCTGTCCACCAAGGACGTGTGCGTGCCCCCCCGCCGCCAG
GAGCTGTGCCTGGGCAACATCGACCGCATCTACGACAAGAACCTGCTGAT
GATCAAGGAGCACATCCTGGCCATCGCCATCTACGAGTCCCGCATCCTGA
AGCGCAAGTACAAGAACAAGGACGACAAGGAGGTGTGCAAGATCATCCAG
AAGACCTTCGCCGACATCCGCGACATCATCGGCGGCACCGACTACTGGAA
CGACCTGTCCAACCGCAAGCTGGTGGGCAAGATCAACACCAACTCCAACT
ACGTGCACCGCAACAAGCAGAACGACAAGCTGTTCCGCGACGAGTGGTGG
AAGGTGATCAAGAAGGACGTGTGGAACGTGATCTCCTGGGTGTTCAAGGA
CAAGACCGTGTGCAAGGAGGACGACATCGAGAACATCCCCCAGTTCTTCC
GCTGGTTCTCCGAGTGGGGCGACGACTACTGCCAGGACAAGACCAAGATG
ATCGAGACCCTGAAGGTGGAGTGCAAGGAGAAGCCCTGCGAGGACGACAA
CTGCAAGCGCAAGTGCAACTCCTACAAGGAGTGGATCTCCAAGAAGAAGG
AGGAGTACAACAAGCAGGCCAAGCAGTACCAGGAGTACCAGAAGGGCAAC
AACTACAAGATGTACTCCGAGTTCAAGTCCATCAAGCCCGAGGTGTACCT
GAAGAAGTACTCCGAGAAGTGCTCCAACCTGAACTTCGAGGACGAGTTCA
AGGAGGAGCTGCACTCCGACTACAAGAACAAGTGCACCATGTGCCCCGAG
GTGAAGGACGTGCCCATCTCCATCATCCGCAACAACGAGCAGACCTCC
EBA-175 RII-N-gly amino acid sequence

(SEQ ID NO: 7)

GRQTSSNNEVLSNCREKRKGMKWDCKKKNDRSNYVCIPDRRIQLCIVNLA
IIKTYTKETMKDHFIEASKKESQLLLKKNDNKYNSKFCNDLKNSFLDYGH
LAMGNDMDFGGYSTKAENKIQEVFKGAHGEISEHKIKNFRKKWWNEFREK
LWEAMLSEHKNNINNCKNIPQEELQITQWIKEWHGEFLLERDNRAKLPKS
KCKNNALYEACEKECIDPCMKYRDWIIRSKFEWHTLSKEYETQKVPKENA
ENYLIKISENKNDAKVSLLLNNCDAEYSKYCDCKHTTTLVKSVLNGNDNT
IKEKREHIDLDDFSKFGCDKNSVDTNTKVWECKKPYKLSTKDVCVPPRRQ
ELCLGNIDRIYDKNLLMIKEHILAIAIYESRILKRKYKNKDDKEVCKIIQ
KTFADIRDIIGGTDYWNDLSNRKLVGKINTNSNYVHRNKQNDKLFRDEWW
KVIKKDVWNVISWVFKDKTVCKEDDIENIPQFFRWFSEWGDDYCQDKTKM
IETLKVECKEKPCEDDNCKRKCNSYKEWISKKKEEYNKQAKQYQEYQKGN
NYKMYSEFKSIKPEVYLKKYSEKCSNLNFEDEFKEELHSDYKNKCTMCPE
VKDVPISIIRNNEQTS

Residues in bold are the mutations used to abolish glycosylation during expression (N3Q, S50A, S195A, T206A, N400Q)

Note that the last 14 residues were found to be truncated during expression (by proteolysis). The crystal structure of the complex shows ordered regions for amino acids 8-601.

Fermentation

Seed Culture

The inoculum culture is prepared using a two-stage seed process of Pichia pastoris. The first stage is initiated by inoculating one vial of frozen glycerol stock (EM31-001) into 125 mL BMGY media in a 500 ml flask and incubated at 250 rpm and 30° C. for approximately 24 hours to a final OD 600 nm of 15-25. The second stage is initiated by transferring 20 mls of the first seed in to 250 mls BMG media in a 2 L Shake Flask and is incubated at 250 rpm and 30° C. for approximately 14 hours to a final OD 600 nm of 15-30.

Main Fermentation

The fermentation media consists of Calcium Sulfate, Potassium Sulfate, Magnesium Sulfate, Potassium Hydroxide, Phosphoric Acid and Glycerol. Post sterilization addition of Trace Salts Solution is necessary. The fermentation consists of four main phases: batch glycerol, fed-batch glycerol, methanol ramp and methanol soak. The batch glycerol phase is the beginning phase which utilizes the initial charge of glycerol as the carbon source. This phase lasts for approximately 24 hours. A sharp DO spike characterizes the end of this phase. The spike indicates the depletion of the carbon source.

The fed-batch glycerol phase is initiated at a set flow (16.6 g/L/hr) immediately following the batch glycerol phase. The fed-batch glycerol phase lasts for 6 hours. During the final two hours of the fed-batch phase, the pH is ramped linearly from 5.0 to 6.0 and temperature was linearly decreased from 30° C. to 24° C.

The methanol ramp phase is initiated immediately following the fed-batch glycerol phase. The methanol is used as a carbon source and as a product inducer. EBA-175 RII-NG is produced as a secreted protein. During this phase, the methanol flowrate to the fermentor is ramped linearly from 1.5 to 8.5 mL/L/hr with a ramping rate of 1 ml/L/hr2.

The final phase of the fermentation is the methanol induction phase. The methanol continues to be used as a carbon source and product inducer. During this phase the methanol is fed to the fermentor at a set rate of 8.5 mL/L/hr for 82 hours. Harvest conditions are to be established at a later time. The fermentation supernatant may be centrifuged and stored at −20° C. if necessary for development of purification conditions. Final EBA-175 RII-NG concentration is approximately 1.4-1.8 g/L fermentation supernatant. The final WCW is approximately 480 g/L.

Process Development and Purification

An extensive process was developed consisting of a tangential flow harvest, CM Hyper DF cation exchange capture step, followed by MEP-Hyper cell HClC chromatography, and a Mustang Q anionexchange chromatography with an SPHP cationexchange polishing step and a ultrafiltration diafiltration concentration step.

Description of Purified Material

The purified EBA-175 RII-NG protein has a molecular mass of 72048 Daltons as determined by mass spectrometry. This is 1561 Daltons less than that of the theoretical value (73609 Daltons). By N-terminal sequencing analysis, purified EBA-175 RII-NG protein possesses the correct N-terminal glycine residue, and there is no evidence to suggest the presence of internal cleavage of the purified RII-NG protein. This discrepancy appears to come from a C-terminal truncation. Based on the predicted sequence, a deletion of 14 amino acid residues (603 DVPISIIRNNEQTS 616) (SEQ ID NO: 7) from the expected C-terminus of EBA-175 RII-NG will result in a truncated molecule with a molecular mass of 72041 Daltons. This experimental value is essentially the same as the predicted molecular mass of the protein having a C-terminal truncation. This was further confirmed by Lys-C peptide mapping of purified EBA-175 RII-NG. Among five different lots tested so far, no evidence of the presence of this intact Cterminal fragment has been identified.

Cloning of Synthetic EBA-175 RII-N-gly- Gene (eba-175 RII-N-gly-) in the P. pastoris Expression Vector pPICZαA

Plasmid 3D7DEF-4 containing the synthetic gene encoding EBA-175 RII-N-gly⁻ (eba-175 RII-N-gly⁻) was restricted with XhoI and XbaI restriction enzymes as per the manufacturers suggestion. The restricted eba-175 RII-N-gly⁻ fragment (˜1.8 kb) was gel purified using the Gel Extraction Kit (QIAGEN, Inc.) and cloned into the XhoI and XbaI sites in pPICZαA. The ligation mix was transformed into the Top 10 E. coli. strain (Invitrogen, San Diego, Calif.). The plasmid pPICZαA/ENTRIIvm/4 containing eba-175 RII-N-gly⁻ gene was selected by restriction analysis and sequence verified. pPICZαA/ENTRIIvm/4 plasmid DNA was prepared using Qiagen Plasmid Maxi Kit (QIAGEN, Inc.), linearized with the restriction enzyme SacI (Life Technology, Gaithersburg, Md.), and transformed into P. pastoris host strain X33 (His4^{+Mut}⁺, Invitrogen, San Diego, Calif.) by electroporation as suggested (Invitrogen manual). P. pastoris transformed with pPICZαA/ENTRIIvm/4 and selected on YPD plates with 100, 200 or 500 g/ml Zeocin. P. pastoris transformants (16) were streaked for single colony separation on YPD+100 _g/ml Zeocin plates.

Recombinant Protein Expression of RII-N-gly⁻ By Shake-Flask Fermentation

Single colonies were tested for expression of RII-N-gly⁻ in BMGY/BMMY media for 96 hours at 30° C. and time-point samples were collected every 24 hours. Expression of RII-N-gly⁻ in culture supernatants at 96 hours was analyzed by Coomassie stain of SDS-PAGE gels. Two clones yielding higher levels of RII-N-gly⁻ were selected for analysis of the complete time-course by Coomassie stain of SDS-PAGE gels and immunoblot with RII specific mAb R217 and rabbit polyclonal IgG. One P. pastoris clone X33/pPICZαA/ENTRIIvm/4/14 was selected as the production clone. The results are documented in the notebook listed above.

Preparation of the P. pastoris clone X33/pPICZαA/ENTRIIvm/4/14 Glycerol Stock

A single colony from the X33/pPICZαA/ENTRIIvm/4/14 YPD+Zeocin plate was inoculated into BMGY and grown overnight at 30° C. as suggested (Invitrogen manual). Sterility of the culture was examined by light microscopy (400×). A glycerol stock (P1) was prepared in 15% glycerol and stored at −70° C.

A P2 glycerol stock was prepared by inoculating 100 _{—}1 of P1 stock into 100 ml of BMGY and grown overnight at 30° C. as suggested (Invitrogen manual) and saved in a final of 15% glycerol and stored at −70° C. P2 glycerol stocks are labeled as follows: X33/pZαA/ENTRIIvm/4/14, P2, 3/15/01.

Protein Crystallization and Derivatization

Form 1 crystals were grown by vapor diffusion (hanging or sitting drop) by mixing 2 μl of the protein solution (15 mg/ml in 10 mM Tris-HCl at pH 7.4, 100 mM NaCl) with 2 μl of a reservoir solution containing 0.265 M ammonium sulfate, 0.1 M sodium cacodylate at pH 6.5, 29% polyethylene glycol 8000.

Form 2 crystals were grown similarly but with a reservoir containing 2.6-2.8 M ammonium sulfate, 0.1 M sodium cacodylate pH 6.5, 0.05-2% polyethylene glycol 750 monomethyl ether and a protein concentration of 12 mg/ml.

Derivatized crystals were obtained by soaking form 1 or form 2 crystals in saturated lithium sulfate containing either 1 mM sodium dicyanoaureate or 1 mM potassium tetrachloroplatinate for 2 hours.

Complex crystals were grown by mixing 2 μl of the protein solution (20-30 mg/ml in 10 mM Tris-HCl pH 7.4, 100 mM NaCl) with 0.4-1.0 μl of a 10 mM α-2,3-sialyllactose (Sigma) solution and 2 μl of a reservoir solution containing 2.7 M ammonium sulfate, 0.1 M HEPES pH 7.5 and 0.1% polyethylene glycol 750 monomethyl ether.

Data Collection

Native crystals were cryo-protected by transferring into a solution of saturated lithium sulfate and flash freezing in liquid nitrogen. Data were collected at beamlines X26C and X25 at the National Synchrotron Light Source (NSLS) at Brookhaven National Laboratory (BNL).

For derivatives, crystals were flash frozen directly from the soak solution prior to data collection.

Two gold derivatized form 1, one platinum derivatized form 1 and one gold derivatized form 2 crystals were used for data collection. Multi-wavelength anomalous dispersion data were collected at 4 wavelengths for the gold derivatives and 3 wavelengths for the platinum derivatives. Data were collected at beamline X25. All data collection statistics are shown in Table 1 (form 1) and Table 2 (form 2).

Data reduction statistics for the complex indicated a loss of a crystallographic two-fold had occurred upon ligand binding. This resulted in a conversion from a tetragonal P4₁22 to an orthorhombic C222₁ space group (see Table 3). The abolished two fold relates the two monomers of the dimer, which is a non-crystallographic two fold in the complex.

Structure Solution and Analysis

Four gold sites were identified by SnB (Weeks and Miller, 1999) and confirmed by SHELXS (Sheldrick et al., 1993) with data collected at the absorption peak for a form 1 gold derivative. Following phase refinement with SHARP (de la Fortelle and Bricogne, 1997) platinum sites in form 1 were determined by cross-phasing with CNS (Brünger et al., 1998). Phase refinement with the form 1 native and derivative data (4 Au wavelengths, 3 Pt wavelengths with SHARP) allowed the building of a poly-alanine model comprising ˜60% of the backbone in O (Jones and Kjeldgaard, 1997). This model was separated into two halves and used to solve the structure of form 2 by molecular replacement (AMoRe (Navaza and Saludjian, 1997)). Four low occupancy gold sites were then identified by difference Fourier for a form 2 gold derivative. Simultaneous cross crystal averaging, density modification and phase extension (DMMULTI-CCP4 (CCP4: Collaborative Computational Project No. 4, 1994)) using both form 1 and 2 native data and form 1 and 2 initial phases improved the density to allow ˜90% of the backbone and 60% of sequence to be assigned unambiguously. Iterative SIGMAA (CCP4) weighting, RESOLVE (Terwillliger, 2000) prime-and-switch density modification and model building (O) allowed unambiguous assignment of 95% of the sequence. Phasing statistics are shown in Tables 1 and 2.

Refinement of the model and re-building was performed with CNS, Reduce and Probe (Word et al., 1999), MolProbity (Lovell et al., 2003) and O. Final refinement statistics are shown in Table 3. The final model contains residues 8-163, 166-508 and 513-596 of RII, 326 water molecules and 9 sulfates and one chloride ion with an R_{work}/R_{free} of 0.232/0.280. Protein volumes were calculated from the RII structure using VOIDOO (Kleywegt, G. J. and Jones, T. A., Acta Cryst., D50: 178-185 (1994)).

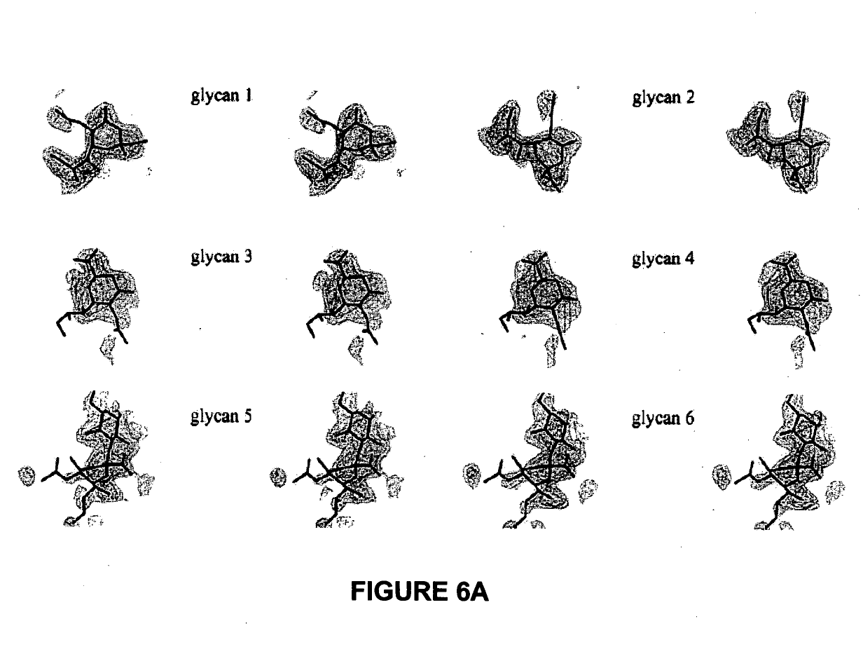

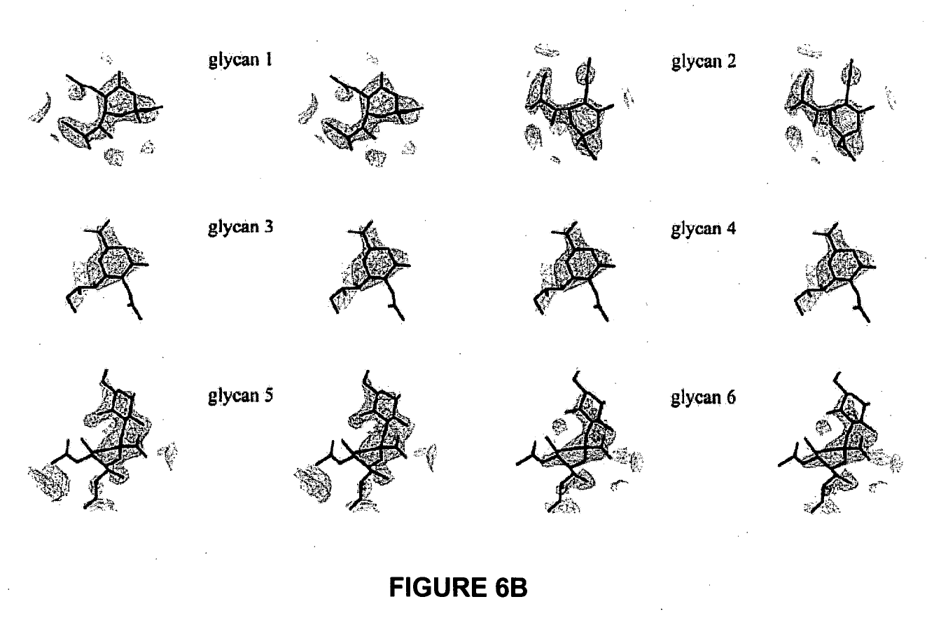

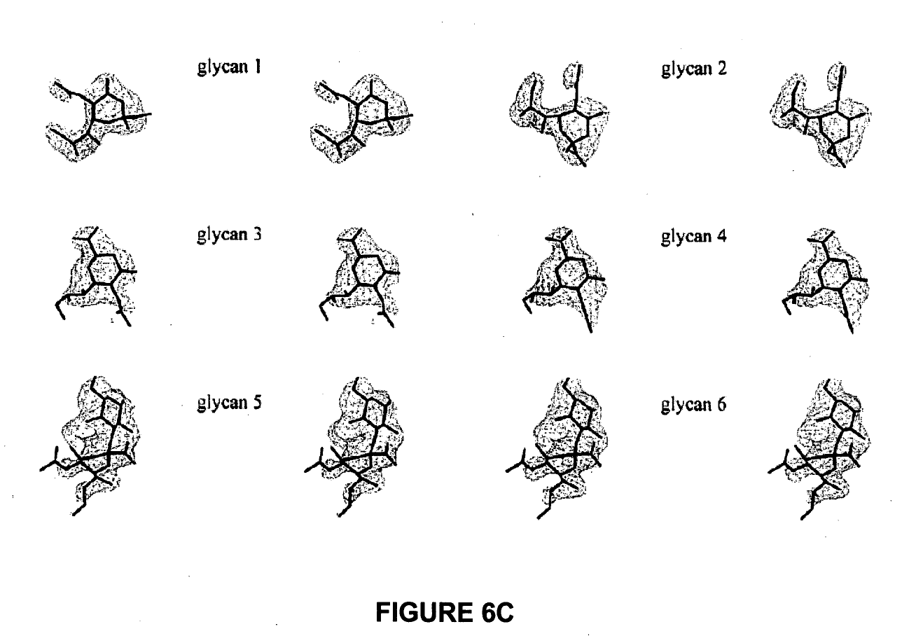

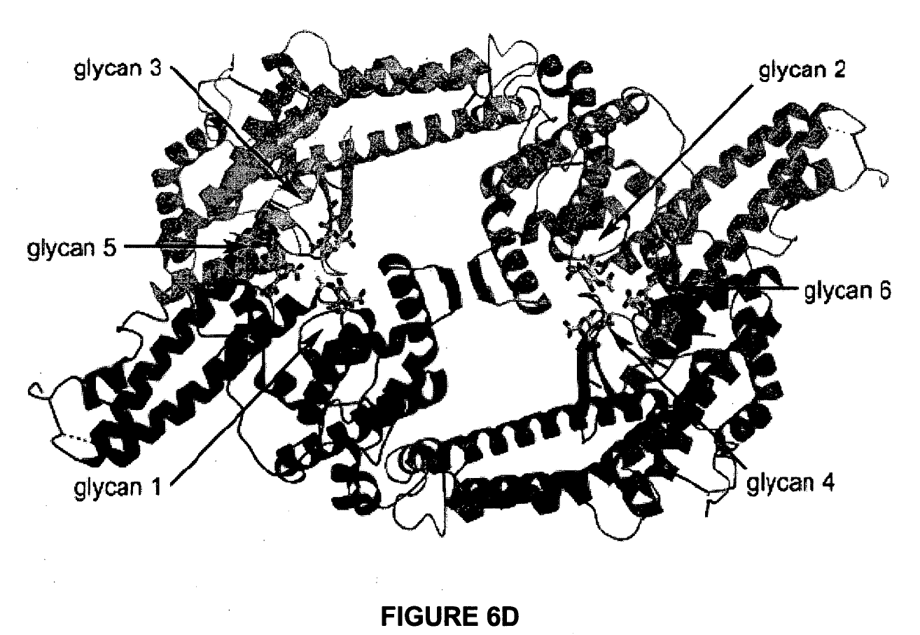

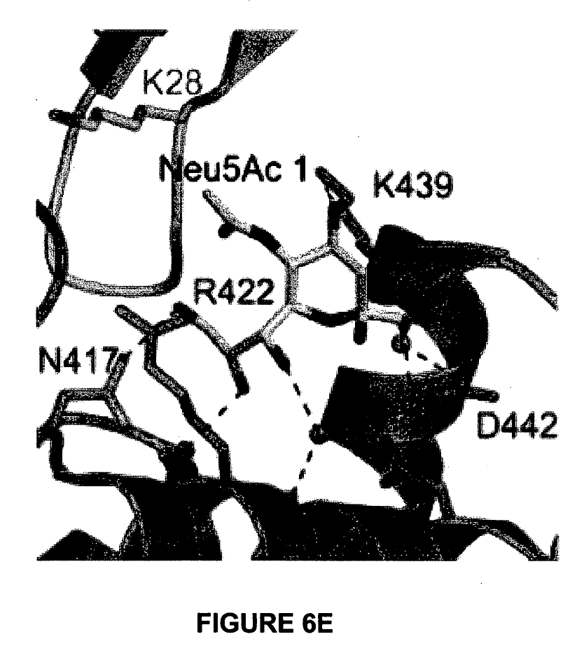

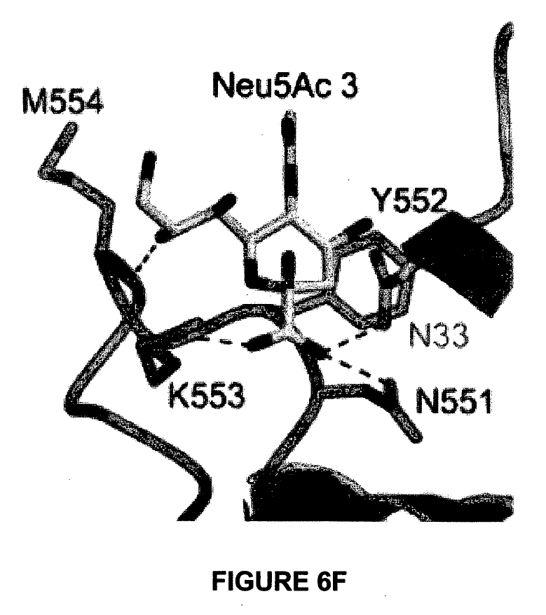

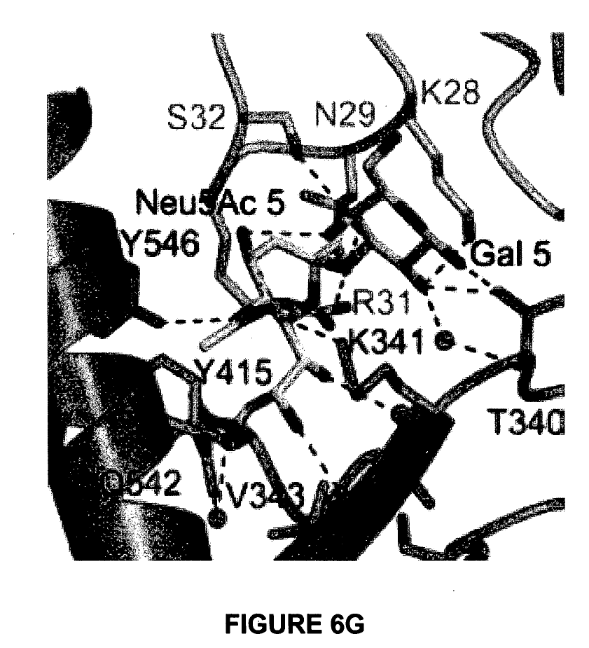

The sialyllactose complex was solved by molecular replacement (AMoRe) using the refined form 2 structure without the solvent molecules as a search model. Two monomers were found in the asymmetric unit. Initially, TLS followed by tightly-restrained NCS refinement with REFMAC5 was followed by model rebuilding and the addition of solvent molecules with O. The locations of the six glycans were identified by visual inspection of F_{obs}-F_{calc} electron density maps, calculated with CNS and shown in FIG. 6A. Final refinement statistics are shown in Table 3. This protocol was chosen based on careful monitoring of the R_{free}. The final model contains two monomers of RII each composed of residues 8-163, 166-508 and 513-601, 599 water molecules, 16 sulfates and two chlorides with an R_{work}/R_{free} of 0.216/0.232. A possible model for the glycans was also built and the resulting 2F_{obs}-F_{calc} electron density maps for the glycans are shown in FIG. 6B. In addition, a simulated-annealed F_{obs}-F_{calc} omit map (FIG. 6C) was calculated by omitting the glycans as well as a 2 Å sphere around them. Though the general placement of these glycans was obvious, a precise atomic model was not possible due to the relatively limited resolution. In addition, due to only partial occupancy of the glycans (0.4-0.6), the protein had to be modeled using alternate conformations for side chains in bound and unbound states. Less than ideal density is not unusual for carbohydrates at this resolution.

Mutagenesis and Red Blood Cell Binding (Rosette) Assays

Single amino acid mutations were introduced in RII, previously cloned into plasmid pRE4, by the Quickchange method (Stratagene). The entire RII sequence for each mutant was sequenced, to ensure accuracy of mutagenesis. Fresh monolayers of COS-7 cells cultured on cover slips in 3.5 cm-diameter wells, were transfected with 2 μg of Qiagen-purified plasmids with the use of calcium phosphate precipitation. Forty-eight (48) hours after transfection one cover slip was removed from each well for immunofluorescence assays to assess expression. Cells were fixed with 2% formaldehyde in phosphate buffered saline, and immunoflourescence observed using standard methods (Sim, B. K. et al., J. Cell. Biol., 111: 1877-1884 (1990)). Rosetting assays were preformed on the remaining coverslips in the 3.5 cm-diameter wells. 200 μl of 10% hematocrit erythrocytes in media were added to each well, mixed and incubated for 2 hours at 37° C. The COS cells were washed three times with phosphate buffered saline to remove non-adherent cells prior to visualization and scoring.

Figure Preparation

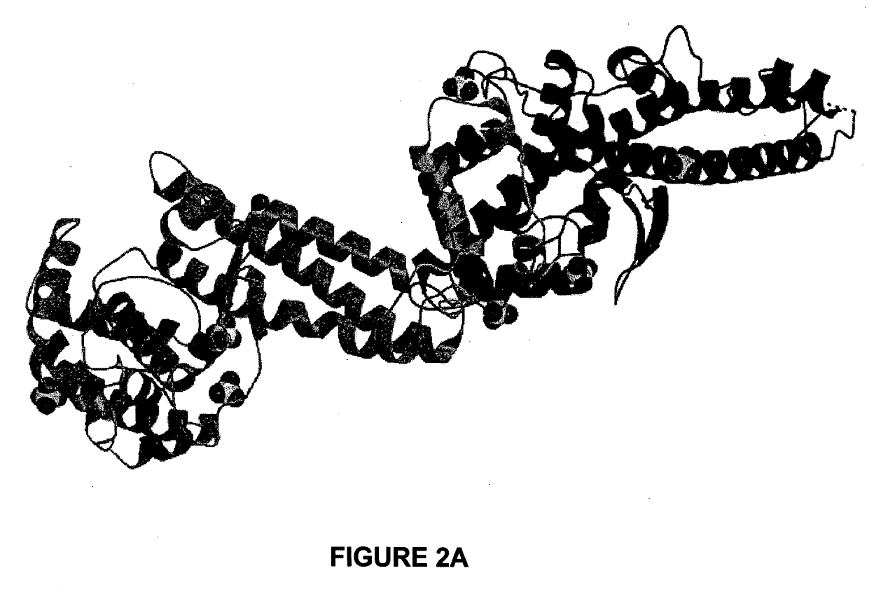

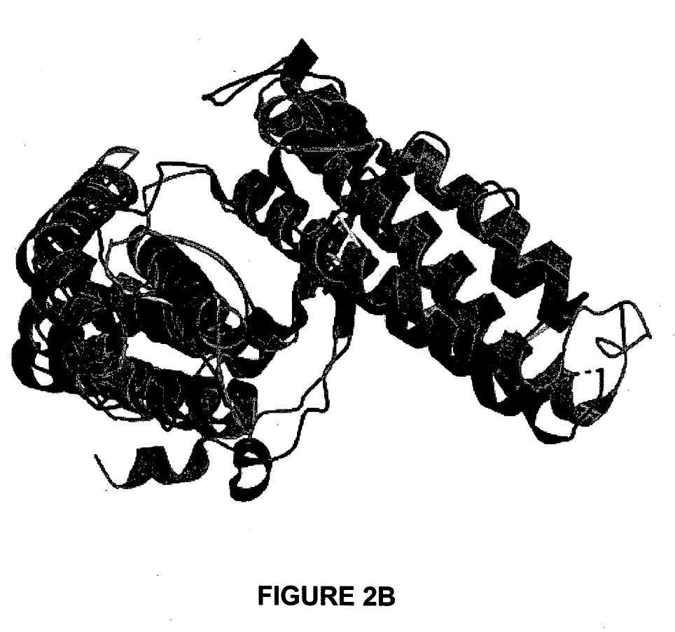

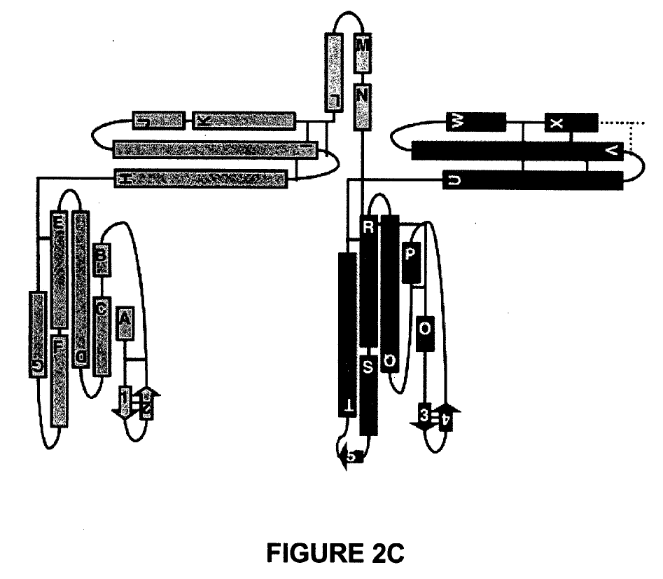

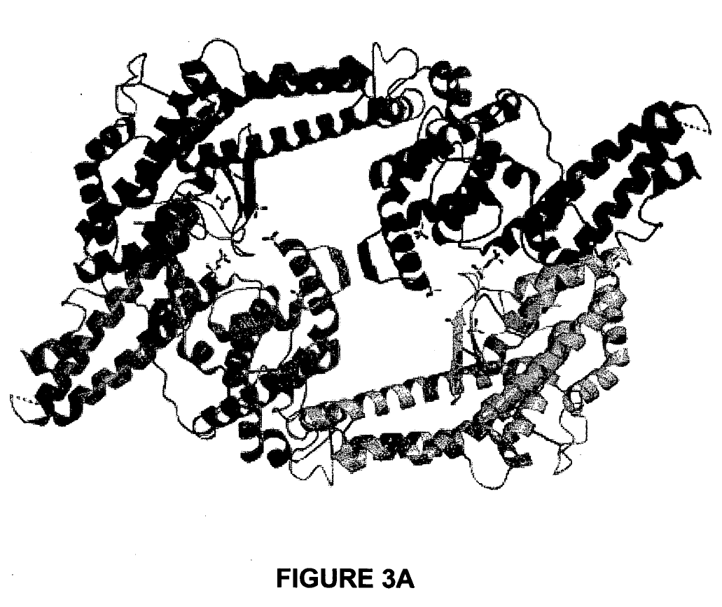

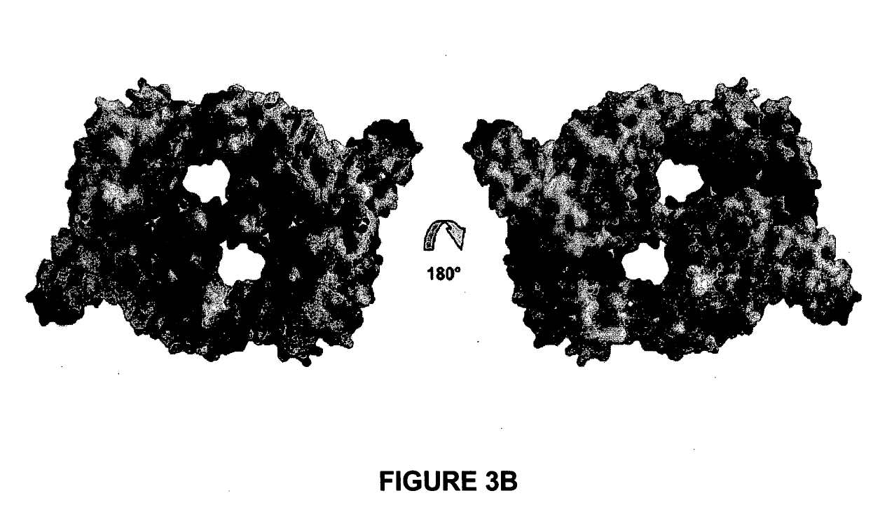

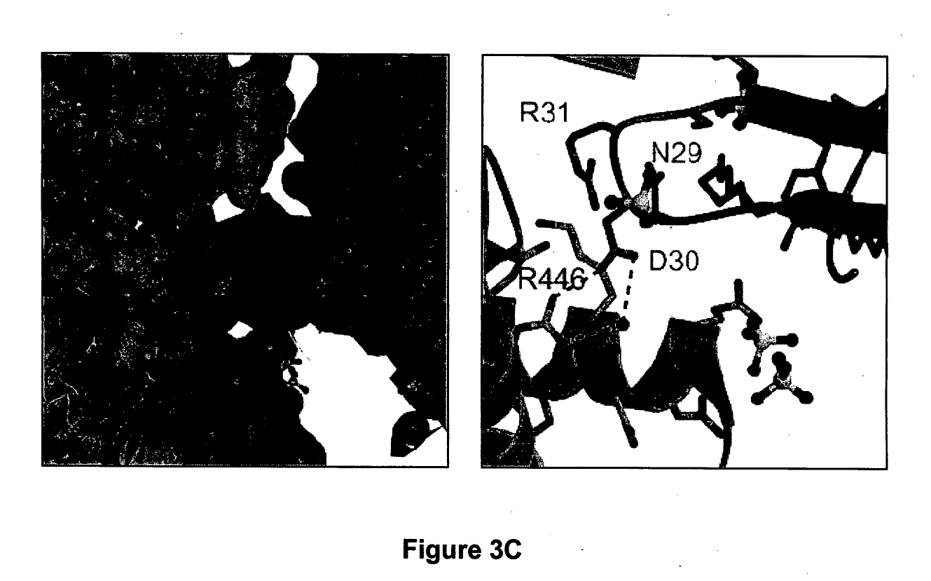

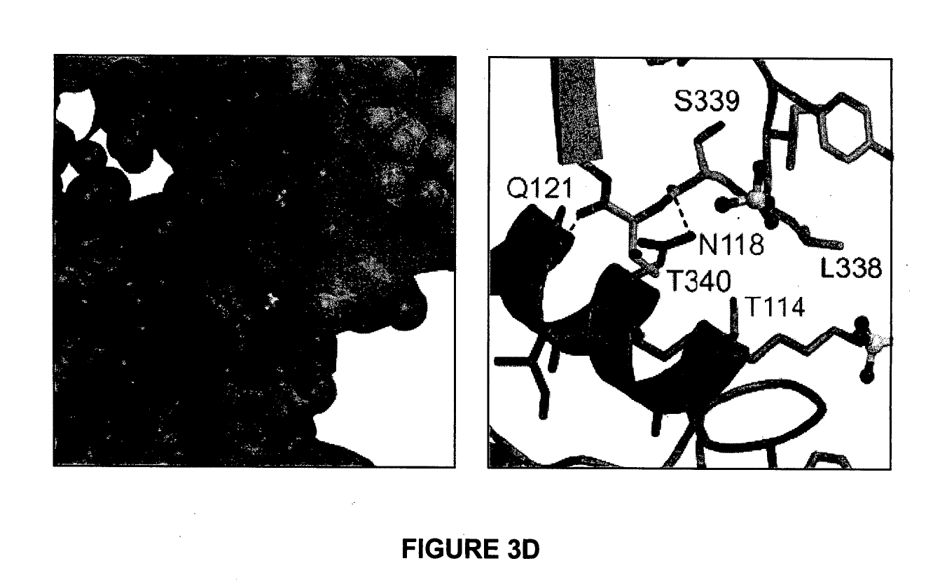

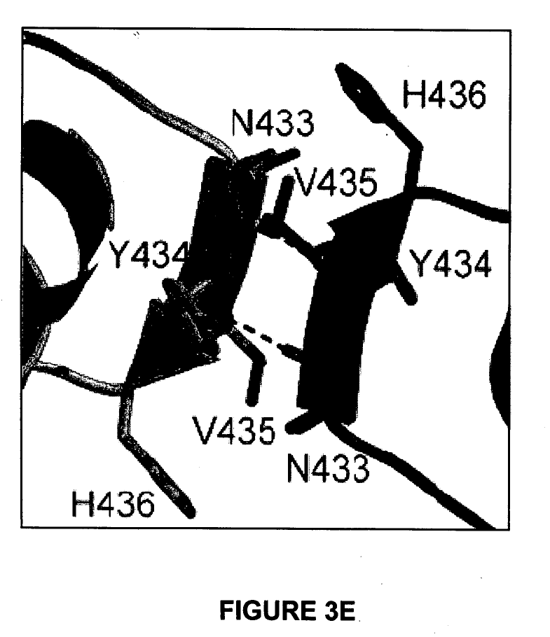

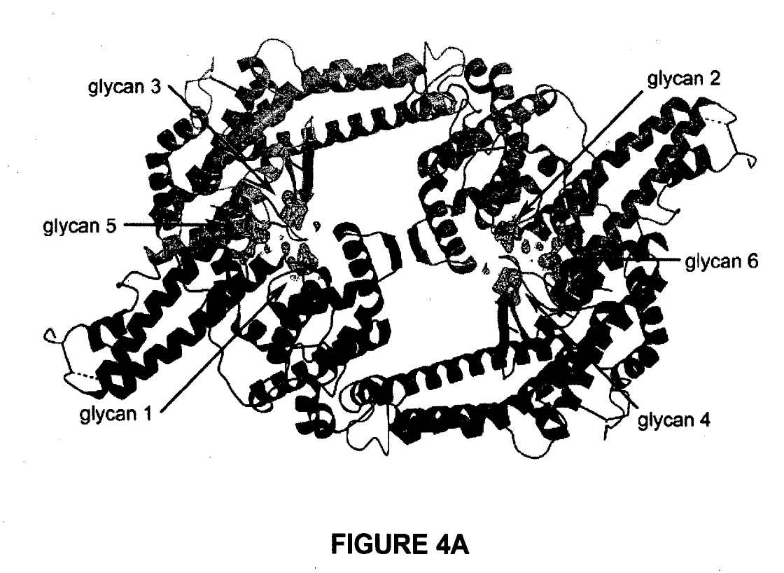

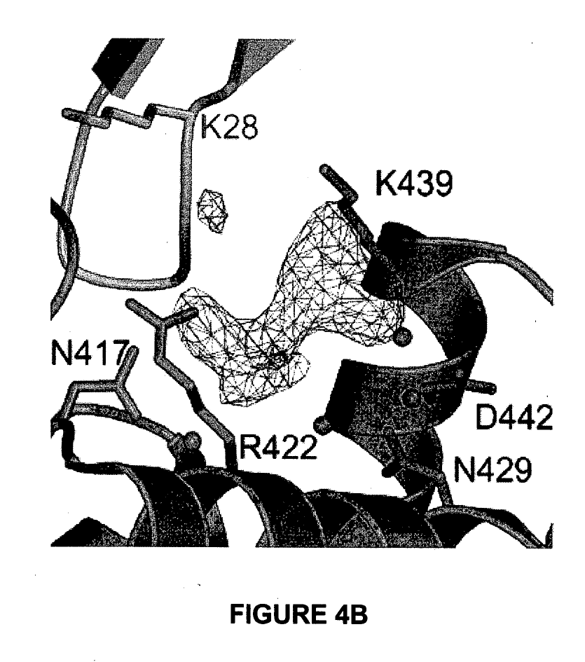

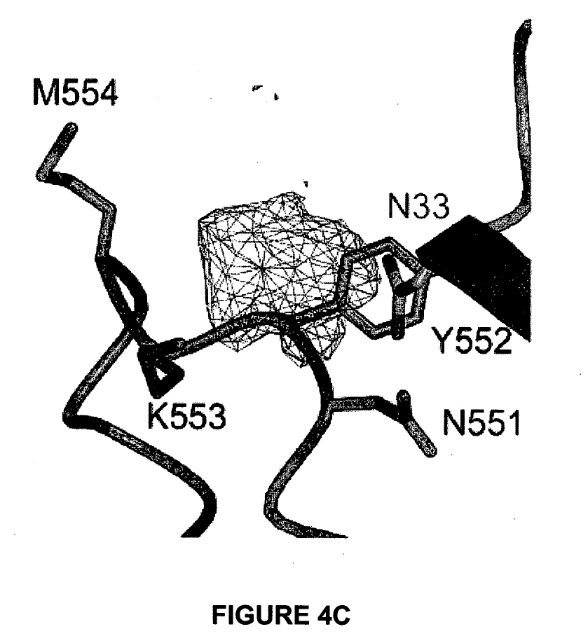

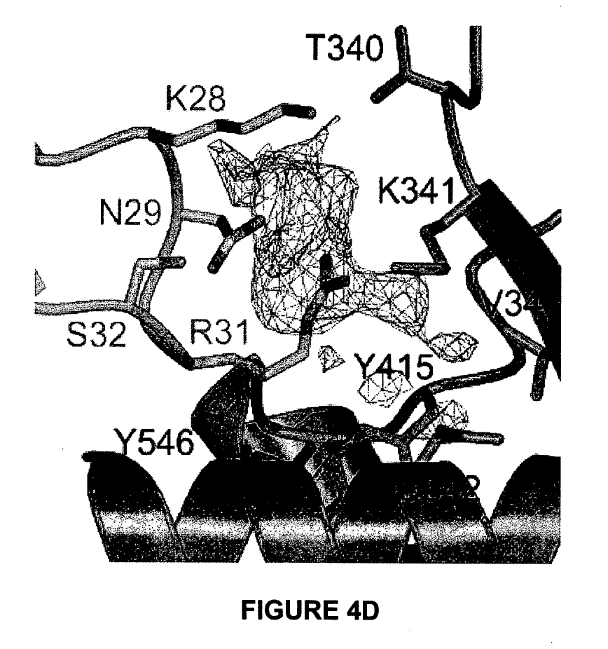

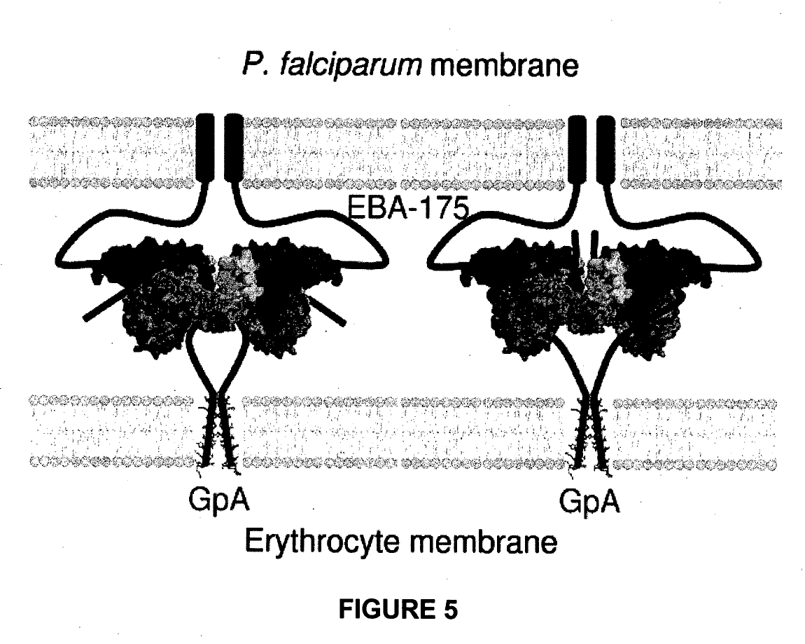

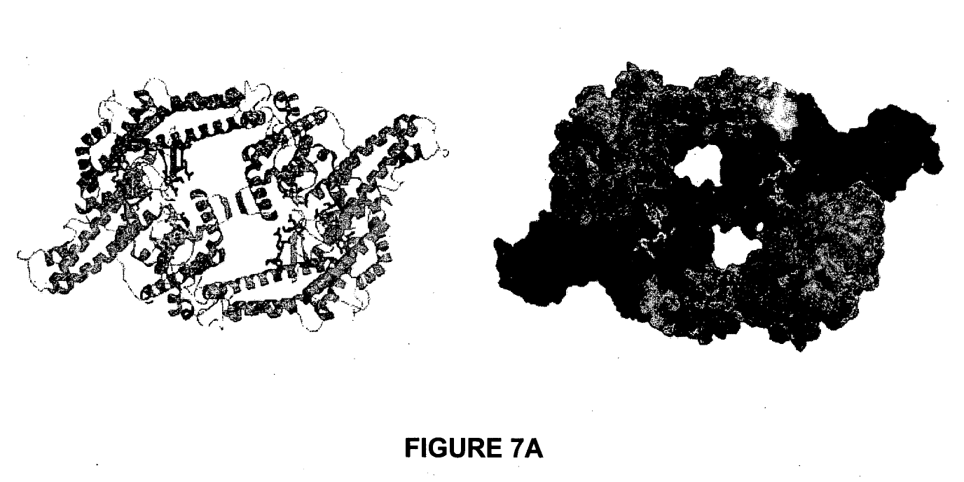

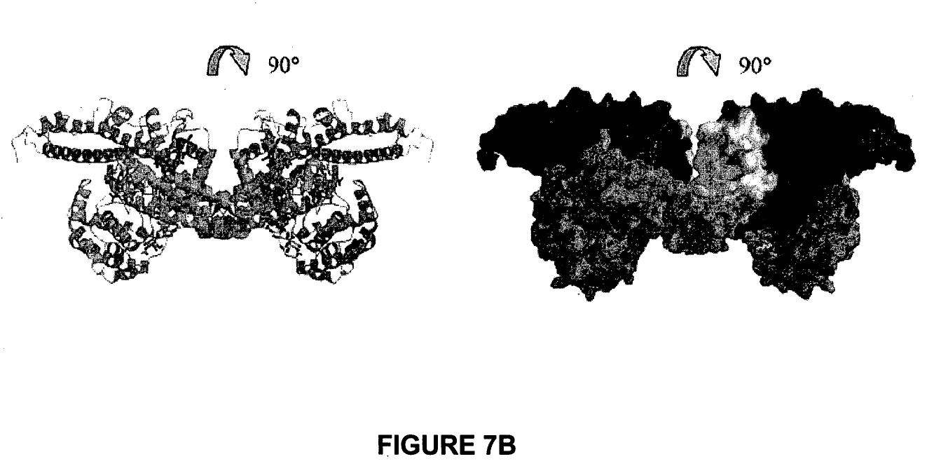

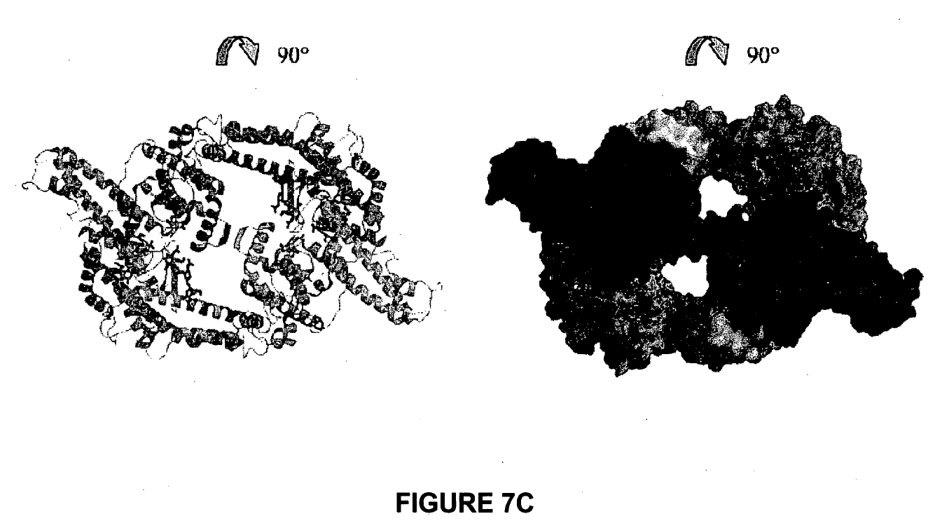

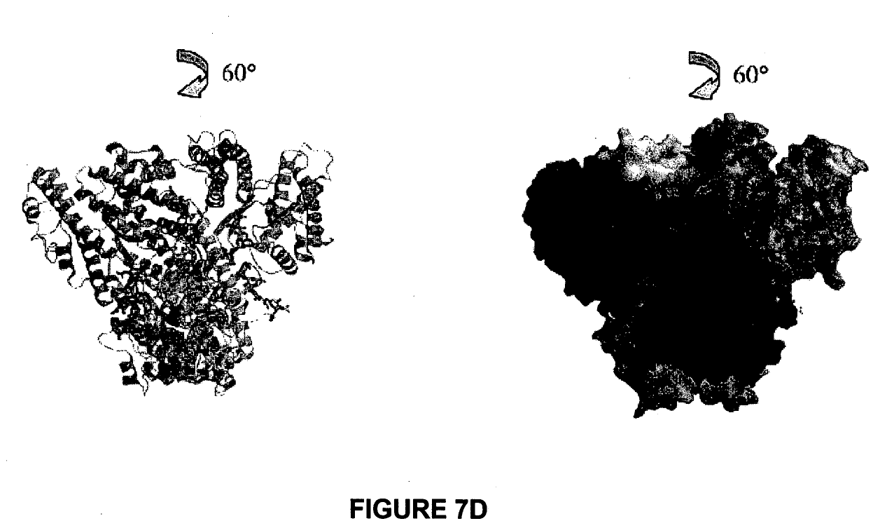

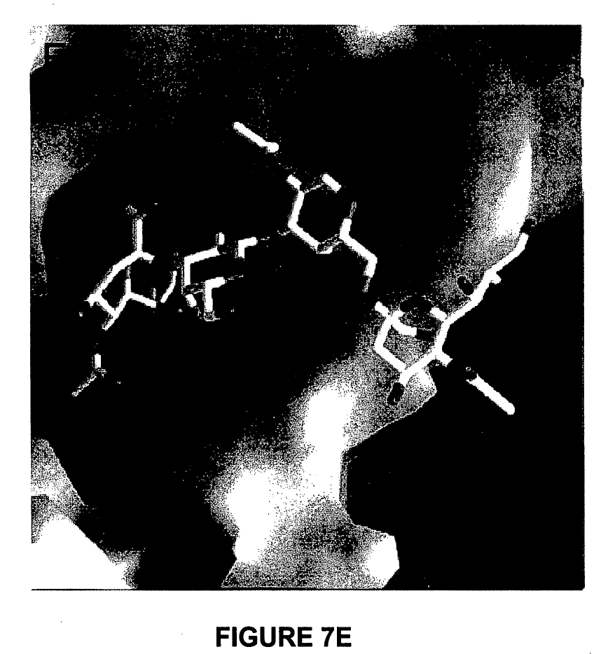

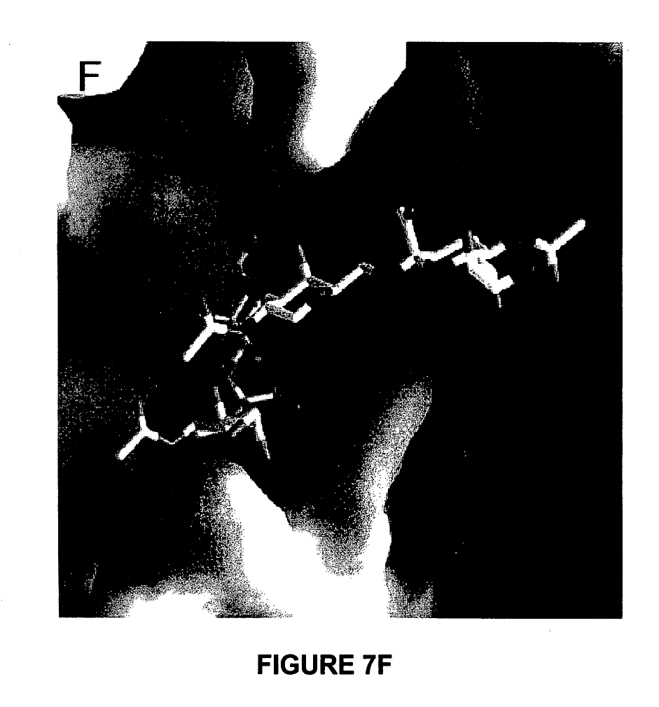

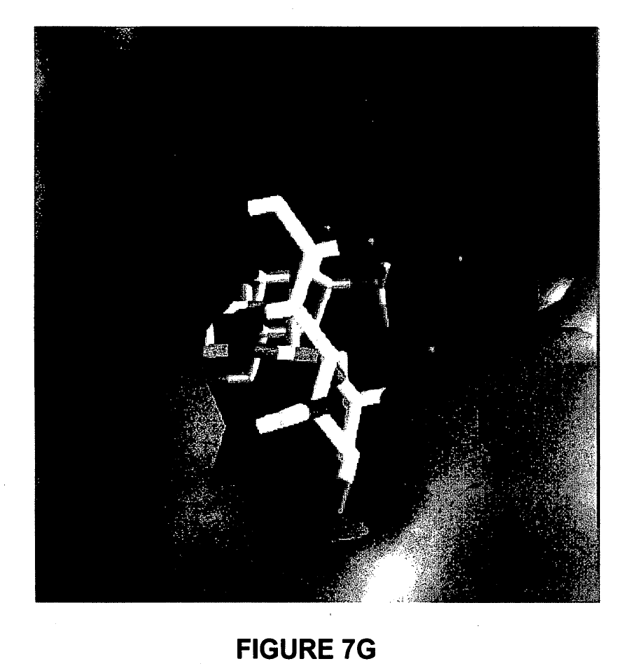

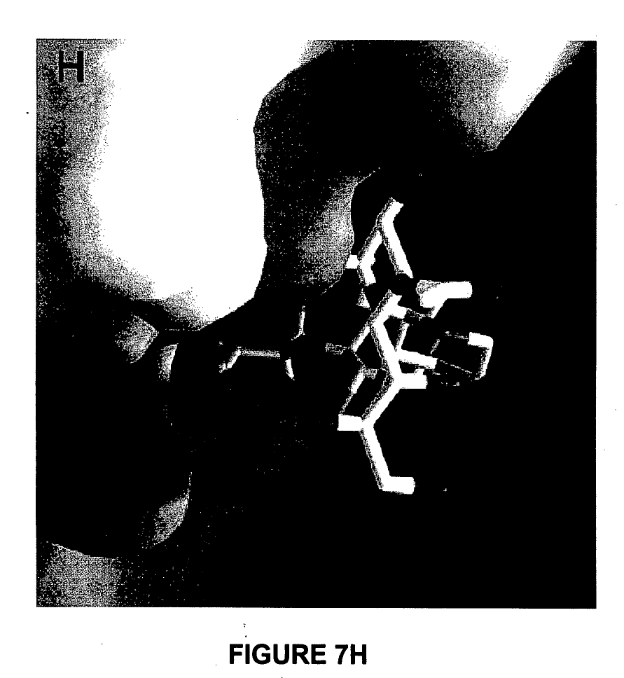

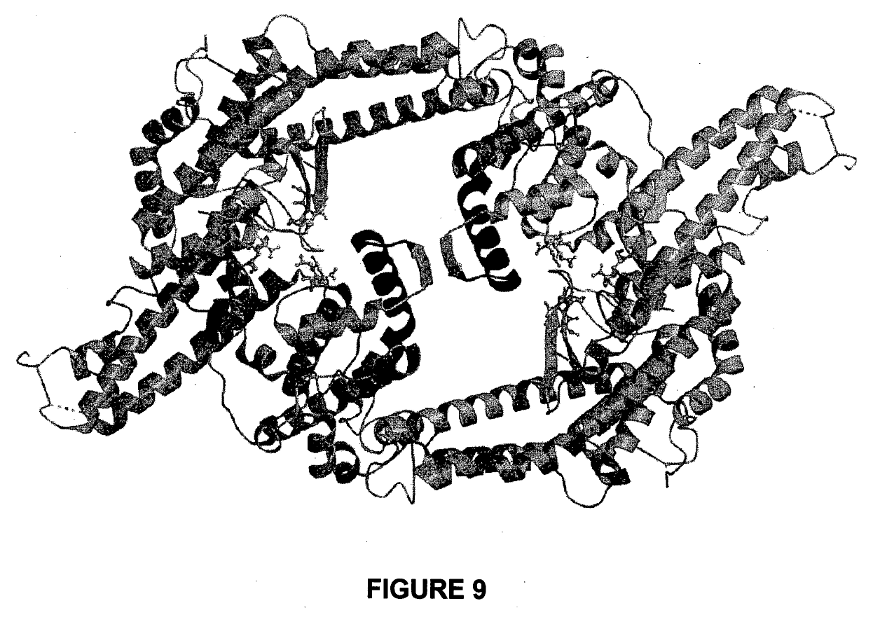

FIG. 2 (Panels A and B), FIG. 3 (Panels A and C-E), FIG. 4, portion of FIG. 5, FIG. 6, left panels of FIGS. 7A-7D and FIG. 9 were prepared using MolScript (Kraulis, P. J., J. Appl. Cryst., 24: 946-950 (1991)), Raster 3D (Bacon, D. J. and

Anderson, W. F. A, J. Molec. Graphics, 6:219-220 (1988); Merritt, E. A. and Murphy, M. E. P., Acta Cryst., D50: 869-873 (1994)) and POVScript+(Fenn, T. D. et al., J. Appl. Cryst., 36: 944-947 (2003)). FIG. 3 (Panels B, C-left, and D-left), right panels of FIGS. 7A-7D and FIGS. 7E-7H were prepared with GRASP (Nicholls, A. et al., Proteins-Structure, Function and Genetics, 11: 281-296 (1991)) and PovScript+(Fenn et al., J. Appl. Cryst., 36: 944-47 (2003)).

### EXAMPLE 2

### Results

Overall Structure of RII

Although the native RII domain of EBA-175 is unglycosylated in P. falciparum due to the lack of post-translational modification capabilities for glycosylation (Gowda, D. C. and Davidson, E. A., Parasitol. Today, 15: 147-152 (1999)), expression in Pichia pastoris resulted in a glycosylated protein, which was refractory to crystallization. Crystallization was successfully carried out with a recombinant non-N-glycosylated RII with the 5 putative glycosylation sites mutated (N3Q, S50A, S195A, T206A, N260Q) for which two different crystal forms were obtained.

The unbound RII domain of EBA-175 of P. falciparum crystallized in two distinct crystal forms. The structure of the protein was determined by multi-wavelength anomalous dispersion (MAD). The overall organization of RII in both crystal forms is conserved, with an RMSD for all Cα atoms of 0.9 Å.

The crystal structure of the RII monomer reveals an elongated molecule, composed primarily of α-helices, with two antiparallel α-hairpins and several bound sulfate molecules (FIG. 2A). Circular dichroism measurements confirmed the predominantly α-helical nature of RII in solution. The structure clearly shows the duplication of DBLs by the presence of two very similar domains, F 1 (residues 8-282) and F2 (residues 297-603), with an RMSD of 1.96 Å (calculated by LSQMAN (Kleywegt, G. J., Acta Crystallogr. D. Biol. Crystallogr., 55: 1878-1884 (1999)) for 194 Cα atoms). A linker region composed of three helices (283-317) links these two domains together. A superposition of the F1 and F2 domains is shown in FIG. 2B. These domains possess a novel fold, based on an analysis using the DALI server (Holm, L. and Sander, C., J. Mol. Biol., 233: 123-138 (1993)). Each domain is, in turn, composed of two subdomains. The topology of RII, shown in FIG. 2C, demonstrates the similarity of the two repeats and also depicts the two subdomains of each repeat. All but one cysteine, C273, are involved in disulfide bridges. C273 located at the end of F1. In addition, C598 is not well ordered in the structure of unbound RII but is in a clear disulfide bridge with C513, which located at the end of F2, as seen in the complex described below. All the disulfides bridges are contained within a given subdomain (FIG. 2C). Thus, cysteines are bridged only within their subdomains. The bridges superimpose nearly perfectly between the two domains, and stabilize the RII cysteine-rich repeat fold (F1/F2).

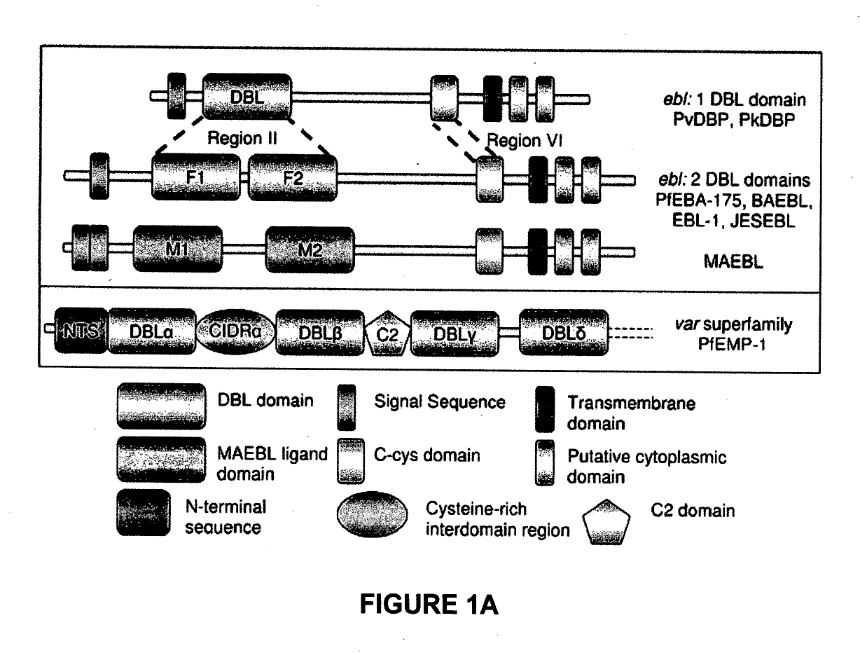

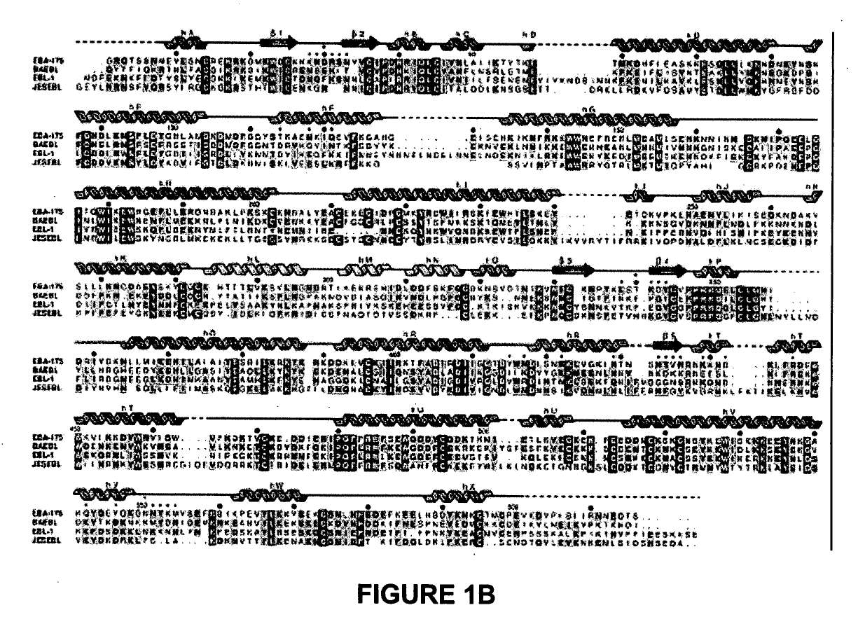

Alignment of EBA-175 RII with other RIIs from the ebl family of proteins (FIG. 1B) reveals 34 invariant residues. With the exception of Q43, W330, W450, W458 and W528, all the invariant residues are involved in fold stabilization (such as hydrophobic packing interactions, hydrogen bonding or salt bridge formation), further contributing to the stability of the DBL domain fold. As with the disulfide bridges, a large number of the invariant residues interact almost exclusively with other residues within their subdomains. Exceptions to this are a salt bridge between R349 and E488, hydrogen bonding between D414 and Q481 and hydrophobic interactions between W330 and W489 and between R439 and W485. These exceptions are all involved in interactions between the two subdomains of F2.

The RII Dimer—A Handshake Interaction

RII crystallizes as a dimer where two symmetrically related RII molecules interact extensively with each other in an anti-parallel fashion resembling a handshake (FIG. 3A). The F1 domain of one monomer interacts mostly with the F2 domain of the second monomer and vice versa. This dimeric interaction is conserved in both crystal forms of RII. The buried surface area is 1480 Å² per dimer, which is consistent within the expected size for a ‘standard-size’ total buried area of 1600±400 Å² for non-covalently interacting proteins (Lo Conte, L. et al., J. Mol. Biol., 285: 2177-2198 (1999)). Gel filtration studies indicate that RII can be purified in both monomeric and dimeric forms. Dynamic Light Scattering (DLS) analysis shows that RII exists as a dimer with a Stoke's radius of 4.29±0.87 nm resulting in an apparent molecular weight of 160 kDa at high concentrations (˜2 mg/ml). The Stoke's radius estimate is within the limits expected from the crystal structure of the dimer. However, analytical ultracentrifugation experiments at lower concentrations (0.4 mg/ml) indicate RII is predominantly monomeric with a small percentage of dimers. In addition, crosslinking studies with bis-sulfosuccinimidyl show that RII exists in both monomeric and dimeric forms. Thus, a mixture of both species, monomers and dimers, in solution was observed. A surface representation of the RII dimer (FIG. 3B) shows a highly basic protein surface (the calculated pI is 8.8) and reveals the presence of two channels that span the dimer. Each channel is composed of surfaces from both monomers. Most of the channel-lining residues, about two thirds, come from the two F2 domains of the dimer.

The two α-hairpins of F1 and F2 form structural modules we termed ‘β-fingers’ that mediate intermolecular interactions. Residues of the F1 β-finger insert into a cavity in F2 (FIG. 3C), and residues of the F2 β-finger insert into a cavity in F1 (FIG. 3D). Some key interactions are a salt-bridge between D30 (F1 β-finger) and R446 (F2 cavity) and van der Waals interactions between T114 (F1 cavity) and L338 and T340 (F2 β-finger). The dimer is further stabilized by H-bonds between the N118 side chain (F1 cavity) and the backbone carbonyl of S339 of the F2 β-finger, and between the carbonyl of T340 (F2 β-finger) and the amide group of Q121 (F1 cavity).

The third intermolecular interaction is the formation of a two-strand anti-parallel 1-sheet between identical F2 residues (N433-H436-β5) of each monomer (FIG. 3E). This b-sheet is found in the center of the dimer, and separates the two channels.

Several well-ordered sulfate molecules are observed at the same positions in both crystal forms. Interestingly, these are bound exclusively in the channels and on one face of the dimer (FIG. 3B), which is termed herein as “bottom” surface for the purpose of discussion.

Receptor Binding Sites

In order to locate receptor binding surfaces and study the binding mode of RII to the GpA receptor, RII with the glycan a-2,3-sialyllactose were co-crystallized. This glycan contains the essential component, Neu5Ac(a-2,3)Gal, of the receptor glycan that is required for binding (Neu5Ac denotes sialic acid). The complex crystallized in a third, related crystal form (form 3), with one dimer in the asymmetric unit (see Materials and Methods). The protein structure is largely unchanged (RMSD 0.39 Å), though there is a slight reduction in the hinge angle between F1 and F2 in the complex. In addition, residues 597-601 that were disordered in the unbound structure are ordered in the complex. This segment includes C598, which is clearly seen in a disulfide bond with C513. All but two of the sulfates were observed as well.

Six glycan binding sites are observed in the RII dimer, all at the dimer interface (FIG. 4 and FIG. 6). Four of these, 1-4, are inside the channels. The other two, 5 and 6, are exposed to the top surface and are separated from the channel by the β-fingers and by glycans 1 and 2. There is also a sulfate between glycan positions 1 and 5 and between 2 and 6. Glycan positions 1 and 2, 3 and 4, and 5 and 6 are related by an approximate two-fold axis.

All six glycans make contacts with both monomers implicating dimerization as important for receptor binding (FIG. 4B, FIG. 6D, Table 2?). Glycans 1 and 2 contact residues N417, R422, N429, K439 and D442 of one monomer and K28 of the other monomer. Glycans 3 and 4 contact residues N550, N551, Y552, K553 and M554 from one monomer, and N33 from the other. Glycans 5 and 6 contact residues T340, K341, D342, V343, Y415, Q542 and Y546 of one monomer, and residues K28, N29, R31 and S32 of the second monomer. The full O-glycan of GpA, Neu5Ac(a2-3)Gal (NeuAc(a-2,6)Gal), was modeled the at each of the glycan binding sites observed in the complex to test whether it would fit. Indeed these studies indicate that the full O-glycan could be accommodated in all of these sites with good geometry (FIG. 7).

Functional Analysis

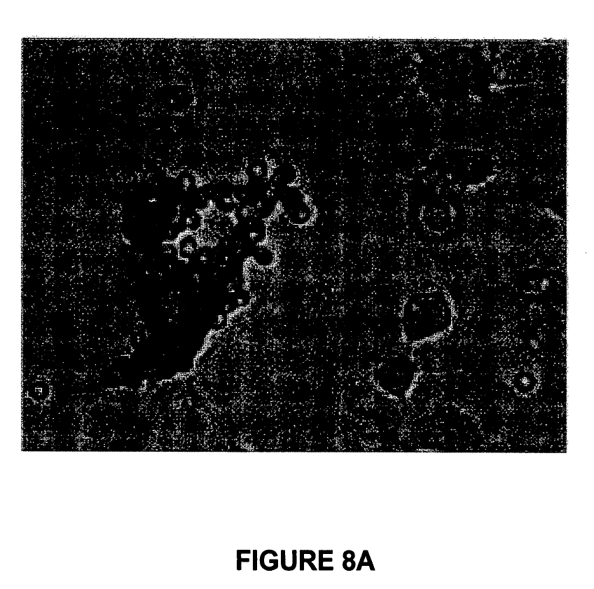

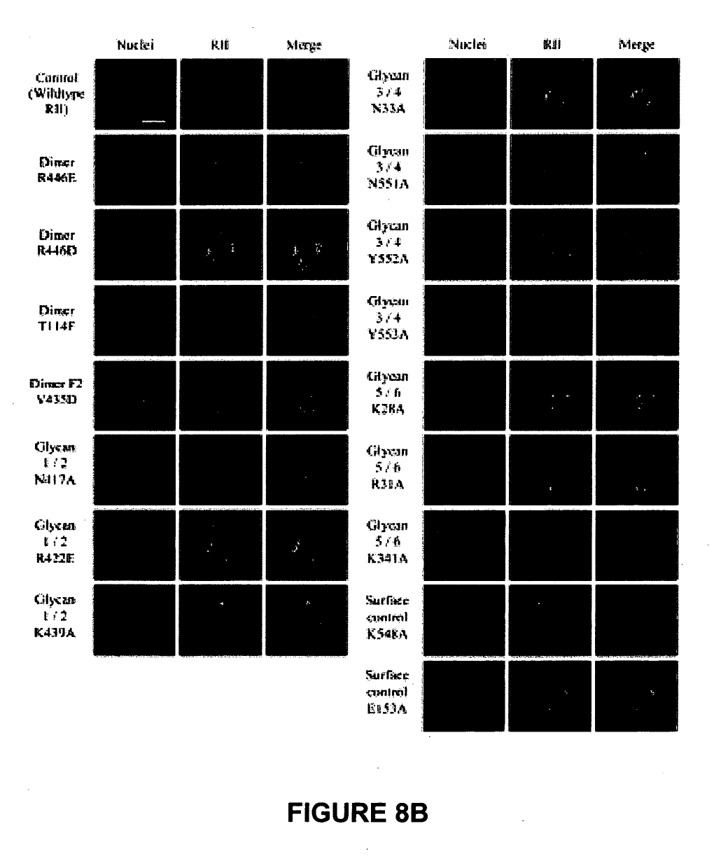

To test the relevance of the dimeric state and glycan binding sites to RII erythrocyte binding, a functional analysis was performed on selected residues involved in these characteristics of RII. RII single point mutants were individually expressed on the surface of COS cells and tested for their ability to bind normal human erythrocytes (Table 6, FIG. 8) (Sim et al., 1994).

Residues involved in the dimeric interaction include D30 and R446, which form a direct salt bridge as well as a water-mediated interaction between the monomers (FIG. 3C); T114, which forms van der Waals interactions with side chains of residues L338 and T340 (FIG. 3D); and backbone atoms in N433-H436 of both monomers that form an antiparallel b-sheet (FIG. 3E). To disrupt the D30/R446 salt bridge, R446 was mutated to a glutamate (R446E) and an aspartate (R446D). Both mutants demonstrated reduced binding to erythrocytes (Table 6). Steric disruption of interactions with T114 by mutation to phenylalanine (T114F) resulted in a reduction in binding efficiency (Table 6). Since the interactions that form the antiparallel b-sheet in the center of the dimer are all backbone interactions, V435 was mutated to aspartate (V435D) to introduce charge repulsion. Again, a reduction in binding is observed (Table 6). Therefore, RII dimerization is important for erythrocyte binding.

Several residues involved in glycan binding were also selected for mutagenesis. N417, R422 and K439 are involved in interactions with glycans 1 and 2. Mutation of these residues result in reduced binding (Table 6). Note that, unlike the others, R422 is a severe mutation to glutamate, which may explain its stronger effect. N33, N551, Y552 and K553, which are all involved in interactions with glycans 3 and 4, were mutated to alanine. Each reduced binding to different extents. Finally, K28, R31 and K341 interact with glycans 5 and 6, and mutation of these residues to alanine also resulted in varying degrees of reduced binding. The relative levels of reduction in binding roughly correlate with the number of glycan contacts made by each residue (Table 6, FIG. 6D, Table 10).

This analysis suggests that all six glycan binding sites are important for RII binding to GpA, as mutations that are predicted to disrupt binding to the glycans observed in the structure of the complex prevent binding of RII to red blood cells. The reduction or lack of binding of any by these mutants was not due to inappropriate expression as expression levels were comparable to wild-type RII as shown by immunofluorescence (FIG. 8). As a negative control, two residues on the outer surface of the RII dimer that are not predicted to be involved in dimerization or glycan binding were changed to alanine, and these show full binding activity (Table 6).

Model For Erythrocyte Binding

GpA exists as an erythrocyte transmembrane dimer with two heavily glycosylated extracellular domains per dimer. The crystal structure of RII, the subject of this study, reveals a dimer with two channels. In addition, six glycan binding sites per dimer of RII were identified, four of which (1-4) are accessible from the channels and the other two (5 and 6) are accessible through a cavity on the “top” surface of RII.

The extracellular domain of GpA inhibits RII binding to erythrocytes, implicating this region of GpA as the receptor for RII (Sim et al., 1994). The presence of two channels in the center of the RII dimer along with the location of the glycans in the sialyllactose complex suggests a mode for GpA binding (FIG. 5). Since the glycans are bound at the dimer interface and make contacts to both monomers, we reason that the RII dimer might assemble around the dimeric GpA extracellular domain and that dimerization of RII may occur upon ligand-receptor binding at the erythrocyte surface during the invasion process. Moreover, binding-induced dimerization is consistent with the observation that glycan 5/6 is bound in a deep elongated pocket created by both monomers. Although this pocket is accessible through a cavity on the top surface of the dimer, the glycan is too bulky to enter without some movement of the protein. On the other hand, GpA binding is straightforward if monomers would assemble around it. Thus, any preexisting dimers would dissociate to allow for receptor binding. This scenario is consistent with both analytical ultracentrifugation and crosslinking studies, mentioned above, that indicate the predominance of monomers in solution. In addition, it should be noted that in a separate study, recombinant non-N-glycosylated RII in phosphate buffered saline at pH 7.4 manufactured for clinical use is a stable monomer. The remaining domains of EBA-175 may also influence the assembly of RII around GpA, impacting a conversion from monomer to dimer. Invasion involves dramatic changes in the parasite that would require signaling within the merozoite. It has been shown that the cytoplasmic C-terminal domain of EBA-175 is essential for invasion but not for trafficking of the protein (Gilberger et al., 2003a). This suggests that signaling occurs through the cytoplasmic domain during the invasion process and that this signaling might be triggered following dimerization upon ligand binding.

The channels are 15 Å in diameter at their narrowest, therefore, this region of the channel would be able to accommodate an unglycosylated segment of GpA, such as residues 5-9, 16-21, 27-36 or 38-43. The glycosylated regions of GpA would be bound to the outer segments of the channels and the top face of RII, while an unglycosylated region of GpA would be bound at the center, the narrowest part, of the channel (FIG. 5). Alternatively, the GpA monomers may dock on the outer surface of RII, feeding glycans into the channels (FIG. 5). Since the structure indicates that the glycan binding sites are formed by both monomers, dimerization of RII would greatly enhance binding to GpA. Indeed, mutations of residues involved in dimerization or in putative glycan binding impair the ability of RII to bind erythrocytes (Table 6).

It was shown previously that F2 alone can bind erythrocytes, but that F1 is insufficient (Sim et al., 1994). F2 contributes about two-thirds of the residues that line the channels (see FIG. 3A and FIG. 7). In addition, there are dimeric interactions exclusively between F2 monomers, but F 1 depends on F2 for dimerization (FIG. 3E, FIG. 7). Thus, without intending to be limited by any particular theory, it is believed that F2 alone can form a weak dimer or can be induced to dimerize by the receptor. Moreover, F2 also contributes 75% of the contacts to the glycans observed in the complex with sialyllactose described above. Thus it appears to provide most of the interaction sites for the receptor.

Interestingly, two peptides (355-375 and 435-455) generated from RII that were shown to bind erythrocytes, and inhibit in vitro merozoite invasion (Rodriguez et al., 2000) mapped to residues lining the channels (Supplementary FIG. 4). Peptide 435-455 includes part of the dimerization interface of the cavity of F2, and includes R446, which is shown here to be required for binding to erythrocytes. This peptide also includes residues that line the channels of the dimer and are involved in glycan interactions. Thus, peptide 435-455 can function either by binding to GpA directly, thereby capping GpA on erythrocytes and blocking merozoite invasion, or by binding to EBA-175 RII preventing dimerization. Mutagenesis of this peptide also led to the generation of more immunogenic peptides that protect from challenge with P. falciparum (Guzman et al., 2002).

Implications for the DBL superfamily

Several erythrocyte invasion pathways have been identified in P. falciparum. Some pathways involve DBL containing proteins, in particular those of the ebl family, with the EBA-175/GpA pathway being the dominant chymotrypsin-resistant invasion pathway (Duraisingh et al., 2003 a). Other pathways do not involve DBL containing proteins, such as the reticulocyte binding-like family (Duraisingh et al., 2003b; Kaneko et al., 2002; Rayner et al., 2000; Rayner et al., 2001; Taylor et al., 2002; Triglia et al., 2005). Apart from the cysteine-rich binding domain proteins of P. falciparum, P. vivax and P. knowlesi involved in erythrocyte invasion, the DBL superfamily also includes the highly polymorphic, multicopied var proteins adapted for functions including adhesion and receptor recognition. The structure of EBA-175 RII now allows modeling of these DBL containing proteins and predictions about the alternate DBL-dependent pathways of invasion. Furthermore, models of the DBL domains of PfEMP-1 could reveal conserved regions in the variant PfEMP-1 DBL domains and/or lead to an understanding of PfEMP-1 receptor/ligand interactions. FIG. 1B shows a sequence alignment of the double DBL domain containing RIIs of EBA-175, BAEBL, EBL-1 and JESEBL. Interestingly, the sequence conservation is higher in F2 than in F 1, suggesting a greater selective pressure on F2. This is in accordance with the observation that binding is dependent on F2 to a greater extent than F 1. Most invariant residues including the cysteines are involved in interactions that stabilize the DBL domain fold. In addition, a large number of residues that are involved in Neu5Ac(a-2,3)Gal binding are conserved among five RIIs of the ebl family.

It is interesting that F1 is similar to the single DBL domain of P. vivax and P. knowlesi Duffy binding proteins that have been shown to bind erythrocytes during invasion (Adams et al., 1992; Chitnis and Miller, 1994). There are also similarities in amino acid signatures between F1 and the DBL and CIDR domain of the var gene family that encode proteins responsible for cyto-adherence and antigenic variation. Thus, the three-dimensional structure of the RII DBL domain should be conserved throughout the DBL protein family. The observed amino acid differences may be driven by pressure for immune evasion. Indeed, in a previous study (Liang and Sim, 1997), a strong conservation of both the structure and function of EBA-175 RII was found across laboratory strains and field isolates with only 16 point mutations leading to radical amino acid changes that were few and scattered. Localization of the amino acids that were involved in the point mutations revealed that 75% of them were on the surface of RII arguing that these mutations might have arisen from immune pressure.

Regions of the ebl family that are surface exposed could be used for targeted vaccine design. Loop regions are often good targets for use in immunization due to their inherent flexibility and conformability. Furthermore, the use of residues involved in dimerization or in glycan binding may prove fruitful for targeted vaccine design as described for peptide 435-455. Design of small molecule inhibitors using the glycan binding sites could prove useful as well. Though protein-protein interactions including dimerization have not been traditional targets for drug intervention, using a small molecule targeted to a well-formed cavity as seen in a crystal structure has proven successful, with disruption of the p53-MDM2 interaction as a recent example (Vassilev et al., 2004). The F2 cavity, required for dimerization, which in turn is necessary for EBA-175 function, could serve as such a target.

The present invention has solved the crystal structure of a double-DBL domain from the ebl family of proteins unbound and in complex with a-2,3-sialyllactose. These structures provide a glimpse into the mechanism of erythrocyte invasion by the Plasmodium parasite. They also provide insights into a large superfamily of proteins critical for the pathogenesis and survival of the parasite. Lastly, this study will help the rational design of novel therapeutics that could aid in the desperately needed treatment of malaria.

Structure of EBA-175 RII In Complex With an Fab Fragment

Monoclonal antibodies shown to inhibit invasion of erythrocytes by P. falciparum were raised against the entire EBA-175 RII protein. To elucidate the epitope recognized on RII by the antibodies, we sought to obtain a crystal of the Fab derived from these monoclonal antibodies and RII. Such information allows for the generation of better vaccines. In addition, the location of the epitope may shed light on the functional regions of RII as these antibodies are neutralizing.

Fab Production and Purification

Fab fragments were produced by a commercial protocol (Pierce ImmunoPure Fab Preparation Kit). Briefly, the monoclonal antibodies were incubated with immobilized papain overnight. The supernatant was then subjected to a protein A column, with the Fab fragments flowing straight through the column. It has been reported that for crystallization, treatment of Fab fragments with iodo-acetamide is often necessary to block free thiol groups generated after cleavage that would otherwise lead to non-specific aggregation. The Fab fragments were then dialyzed against PBS to remove free cysteine and then dialyzed against PBS with 2 mM iodo-acetamide for 12 hours to block exposed thiols. The treated Fab fragments were purified by gel filtration on a HiLoad Superdex 200 16/60 column followed by ion exchange on a Mono-S column.

Complex Formation

Complex formation was induced by mixing excess EBA-175 RII with the purified treated Fab fragments, concentrating the sample and incubating at 4° C. for 2 hours. The RII/Fab complex was purified away from the excess EBA-175 RII by gel filtration on a HiLoad Superdex 200 16/60 column.

Tables

Table 1 lists the crystallographic statistics for crystal form 1 of EBA-175 RII.

Table 2 lists the crystallographic statistics for crystal form 2 of EBA-175 RII.

Table 3 lists the refinement statistics for the different data sets collected of EBA-175 RII.

Table 4 lists examples of residues of EBA-175 RII involved in dimerization, sialic acid binding and sulfate binding and examples of mutations.

Table 5 (155 sheets) lists the atomic coordinate data for a dimer of EBA-175 RII as derived from X-ray diffraction.

Table 6 lists the binding efficiency for EBA-175 RII mutants.

Table 7 lists the atomic coordinate data for a dimer of EBA-175 RII in a RII-ligand complex as derived from X-ray diffraction.

Table 8 lists ligand compounds that are used for co-crystal structure.

Table 9 lists compounds that bind to EBA-175 RII in in silico docking using the glycan binding sites defined in the co-crystal structure.

Table 10 lists contact distances for modeled glycans.

LENGTHY TABLE REFERENCED HERE
US20070043208A1-20070222-T00001
Please refer to the end of the specification for access instructions.

LENGTHY TABLE REFERENCED HERE
US20070043208A1-20070222-T00002
Please refer to the end of the specification for access instructions.

LENGTHY TABLE REFERENCED HERE
US20070043208A1-20070222-T00003
Please refer to the end of the specification for access instructions.

LENGTHY TABLE REFERENCED HERE
US20070043208A1-20070222-T00004
Please refer to the end of the specification for access instructions.

LENGTHY TABLE REFERENCED HERE
US20070043208A1-20070222-T00005
Please refer to the end of the specification for access instructions.

LENGTHY TABLE REFERENCED HERE
US20070043208A1-20070222-T00006
Please refer to the end of the specification for access instructions.

LENGTHY TABLE REFERENCED HERE
US20070043208A1-20070222-T00007
Please refer to the end of the specification for access instructions.

LENGTHY TABLE REFERENCED HERE
US20070043208A1-20070222-T00008
Please refer to the end of the specification for access instructions.

LENGTHY TABLE REFERENCED HERE
US20070043208A1-20070222-T00009
Please refer to the end of the specification for access instructions.

LENGTHY TABLE REFERENCED HERE
US20070043208A1-20070222-T00010
Please refer to the end of the specification for access instructions.

Throughout the application, various publications are referenced by author, date and citation. The disclosures of these publications in their entirety are hereby incorporated by reference.

While this invention has been particularly shown and described with references to preferred embodiments thereof, it will be understood by those skilled in the art that various changes in form and details may be made therein without departing from the scope of the invention encompassed by the appended claims.

LENGTHY TABLE
The patent application contains a lengthy table section. A copy of the table is available in electronic form from the USPTO web site (http://seqdata.uspto.gov/?pageRequest=docDetail&DocID=US20070043208A1). An electronic copy of the table will also be available from the USPTO upon request and payment of the fee set forth in 37 CFR 1.19(b)(3).

What is claimed:

1. A polypeptide corresponding to erythrocyte binding antigen 175 (EBA-175) region II (RII), or functional EBA-175 RII, in a crystalline form.

2. The polypeptide of claim 1, wherein the polypeptide has a three-dimensional structure defined by a set of atomic coordinates selected from Table 5.

3. The polypeptide of claim 1 or 2, wherein the polypeptide is in a dimer form.

4. The polypeptide of claim 3, wherein the dimer comprises at least one set of receptor binding sites, wherein the binding sites comprise a first set of binding site(s) comprising amino acid residues N417, R422, N429, K439 and D442 of one monomer and K28 of the other monomer, a second set of binding site(s) comprising amino acid residues N550, N551, Y552, K553 and M554 of one monomer, and N33 of the other monomer, or a third set of binding site(s) comprising amino acid residues T340, K341, D342, V343, Y415, Q542 and Y546 of one monomer and K28, N29, R31 and S 32 of the other monomer, or a combination thereof.

5. A crystal comprising an EBA-175 RII protein having SEQ ID NO: 7.

6. A co-crystal comprising the crystalline form of a complex, wherein the complex comprises the polypeptide of claim 1 in association with at least one ligand compound.

7. The co-crystal of claim 6, wherein the polypeptide comprises six ligand-binding sites.

8. The co-crystal of claim 6 or 7, wherein the polypeptide is in a dimer form.

9. The co-crystal of claim 6, wherein the co-crystal has a space group of C222₁ with unit cell dimensions of about a=145.75 A, b=146.21 A and c=214.74 A and alpha=beta=gamma=90.

10. The co-crystal of claim 6, wherein the polypeptide has a three-dimensional structure defined by the atomic coordinates of Table 7.

11. The co-crystal of claim 8, wherein the dimer comprises at least one set of receptor binding sites, wherein the binding sites comprise a first set of binding site(s) comprising amino acid residues N417, R422, N429, K439 and D442 of one monomer and K28 of the other monomer, a second set of binding site(s) comprising amino acid residues N550, N551, Y552, K553 and M554 of one monomer, and N33 of the other monomer, or a third set of binding site(s) comprising amino acid residues T340, K341, D342, V343, Y415, Q542 and Y546 of one monomer and K28, N29, R31 and S 32 of the other monomer, or a combination thereof.

12. A method for obtaining a Duffy binding like (DBL) domain of erythrocyte-binding like (ebl) protein in crystalline form, comprising mixing a DBL domain solution with a reservoir solution, wherein the DBL domain solution comprises about 1 mg/ml to about 30 mg/ml DBL domain protein, about 1-100 mM of a buffer that can hold a pH value of about 5.5-9.0, and about 0-400 mM of a salt, and wherein the reservoir solution comprises either about 0.1-0.4M ammonium sulfate, about 0.01-0.2 M a buffer that can hold a pH value of about 5.5-9.0, and about 10-35% (w/v) polyethylene glycol or polyethylene glycol monomethyl ether having molecular weight (MW) of about 1,000 to about 20,000, or about 1.5-3.5 M ammonium sulfate, about 0.01-0.2 M a buffer that can hold a pH value of about 5.5-9.0, and about 0.01-10% polyethylene glycol or polyethylene glycol monomethyl ether having molecular weight (MW) of about 100 to about 1,000.

13. The method of claim 12, wherein the DBL domain solution comprises about 15 mg/ml DBL domain protein, about 10 mM Tris-HCl, and about 100 mM NaCl and has a pH value of about 7.4, and wherein the reservoir solution contains about 0.265 M ammonium sulfate, about 0.1 M sodium cacodylate, and about 29% polyethylene glycol 8000 and has a pH value of about 6.5.

14. The method of claim 12, wherein the protein solution comprises about 12 mg/ml DBL domain protein, about 10 mM Tris-HCl, and about 100 mM NaCl and has a pH value of about 7.4, and wherein the reservoir solution contains about 2.6-2.8 M ammonium sulfate, about 0.1 M sodium cacodylate, and about 0.05-2% polyethylene glycol 750 monomethyl ether and has a pH value of about 6.5.

15. The method of any one of claims 12-14, wherein the DBL domain protein is EBA-175 RII.

16. The method of any one of claims 12-14, further comprising incubating the mixture in a closed container at a temperature of about 0° C. to about 37° C. over the reservoir solution to form crystals of the protein.

17. The method of claim 16, wherein the temperature is 17° C.

18. A method for obtaining co-crystals of an EBA-175 RII protein and a ligand that is capable of complexing with said protein, comprising mixing an EBA-175 RII protein solution with a reservoir solution and a ligand solution, and incubate the mixture in a closed container to form co-crystals of a complex of the EBA-175 RII protein and the ligand, wherein the EBA-175 RII protein solution comprises about 1-30 mg/ml of the EBA-175 RII protein, about 1-100 mM of a buffer that can hold a pH value of about 5.5-9.0, and about 0-400 mM of a salt, wherein the ligand solution comprises about 1 nM-100 mM of the ligand, and wherein the reservoir solution comprises either about 0.1-0.4 M ammonium sulfate, about 0.01-0.2 M a buffer that can hold a pH value of about 5.5-9.0, and about 10-35% (w/v) polyethylene glycol having molecular weight (MW) of about 1,000 to about 20,000, or about 1.5-3.5 M ammonium sulfate, about 0.01-0.2 M a buffer that can hold a pH value of about 5.5-9.0, and about 0.01-10% polyethylene glycol or polyethylene glycol monomethyl ether having molecular weight (MW) of about 100 to about 1,000.

19. The method of claim 11, wherein the mixture comprises about 21 μl of the protein solution, about 0.4-11 μl of the ligand solution, and about 2 μl of the reservoir solution, wherein the protein solution contains about 20-30 mg/ml EBA-175 RII protein, about 10 mM Tris-HCl, and about 100 mM NaCl and has a pH value of about 7.4, wherein the ligand solution comprises about 10 mM α-2,3-sialyllactose, and wherein said reservoir solution comprises about 2.7 M ammonium sulfate, about 0.1 M HEPES, and about 0.1% polyethylene glycol 750 monomethyl ether and has a pH value of about 7.5.

20. The method of claim 12 or 18, wherein crystallization is carried out by a vapor diffusion process.

21. A method for computer-based drug design, comprising:
a). obtaining a three-dimensional representation of at least one ligand binding site of an EBA-175 RII protein;
b). superimposing at least one candidate ligand compound on said three-dimensional representation of the ligand binding site;
c). evaluating the binding of said at least one candidate compound and said ligand binding site; and
d). selecting a compound that spatially fits said ligand binding site.

22. The method of claim 21, wherein said three-dimensional representation of the at least one ligand binding site of the EBA-175 RII protein is determined from a crystal or co-crystal comprising the EBA-175 RII protein.

23. The method of claim 21, wherein the ligand is selected from the group consisting of N-acetylneuraminic acid, 2-deoxy-2,3-dehydro-N-acetylneuraminic acid, N-glycolyl-neuraminate, α-2,3-sialyllactose, 2-oxo-2-(4-phenoxy phenyl)ethyl 2-(3-chlorophenyl) 1,3-dioxo-5-isoindoline carboxylate, C₂₇H₂₀F₂N₂O₃, 8-(2-(3,4-dimethoxy-phenyl)-ethylamino)-3-methyl-7-phenethyl-3,7-2H-purine-2,6-dione, C₂₆H₂₀FNO₆, C₃₁H₂₄FN₃O₅S, 2′,3′-dibenzoyluridine, C₂₄H₂₁N₅O₄S, C₂₈H₁₆N₂O₆S, C₂₅H₂₄FNO₆, C₂₇H₂₅FN₂O₆S, and analogs or derivatives thereof.

24. A computer-based method of rational drug design comprising:
a) providing a three-dimensional structure of EBA-175 region II as defined by the atomic coordinates of Table 7;
b) providing a three-dimensional structure of a candidate modulator compound; and
c) fitting the structure of said candidate modulator compound to the structure of said EBA-175 region II of Table 7.

25. The method of claim 24 further comprising:
d) obtaining or synthesizing said candidate modulator compound; and
e) contacting said candidate molecule with EBA-175 region II to determine the ability of said candidate compound to interact with EBA-175 region II.

26. The method of claim 24 further comprising:
d) obtaining or synthesizing said candidate modulator compound;
e) forming a complex of EBA-175 region II and said candidate compound; and
f) analyzing said complex to determine the ability of said candidate compound to interact with EBA-175 region II.

27. A method of detecting and/or identifying a compound that binds to EBA-175 comprising the steps of:
a) using a three-dimensional structure of EBA-175 region II as defined by atomic coordinates of Table 7;
b) employing said three-dimensional structure to design or select a candidate compound;
c) synthesizing said candidate compound; and
d) determining whether said candidate compound is capable of forming a complex with EBA-175 region II or a functional variant thereof,
wherein formation of said complex indicates that said compound binds to EBA-175.

28. The method of claim 27, wherein the candidate compound is designed or selected using computer modeling.

29. The method of claim 27, wherein the candidate compound is designed de novo or designed based on a known compound.

30. The method of claim 29, wherein the candidate compound is an antibody or antibody fragment.

31. A method for screening for a novel drug comprising:
a) selecting a candidate compound by performing rational drug design using a three-dimensional structure determined from the crystal of any one of claims 1 or 2;
b) contacting the candidate compound with an EBA-175 region II protein or a functional equivalent thereof; and
c) detecting the binding potential of the candidate compound for EBA-175 region II or said functional equivalent,
wherein the candidate compound is selected based on its having a greater affinity for EBA-175 region II or said variant than that of a known drug.

32. A method for structure-assisted drug design, comprising:
a) providing a three-dimensional structure of EBA-175 RII; and
b) modifying a pre-designed or pre-selected candidate modulator compound, based on the three-dimensional structure of EBA-175 RII for enhanced binding of said candidate modulator compound with EBA-175 RII.

33. The method of claim 32, further comprising determining whether the binding between the modified candidate modulator compound and EBA-175 RII is enhanced.

34. A method of constructing a three-dimensional molecular model of EBA-175 region II on a computer system comprising the steps of:
a) providing atomic coordinate data according to Table 7; and
b) analyzing said data using a protein modeling algorithm to form a three-dimensional molecular model of the EBA-175 region II.

35. A computer-based method of rational vaccine design comprising:
a) providing a three-dimensional structure of EBA-175 region II defined by the atomic coordinates of Table 7;
b) determining one or more receptor binding site(s) or oligomerization site(s) from said three-dimensional structure of EBA-175 region II;
c) providing an amino acid sequence of a peptide which includes or binds to one or more said receptor binding site(s) and/or oligomerization site(s); and
d) formulating said peptide of step c) as a vaccine composition.

36. A pharmaceutical composition for treating or preventing malaria, comprising one or more compounds that interact or bind to EBA-175 RII protein, wherein said compound is identified by a method of any one of claims 21, 24, 27, 32, and 35.

37. An isolated EBA-175 RII protein having SEQ ID NO: 7.

38. A composition comprising the isolated EBA-175 RII protein of claim 37 in an aqueous solution.

39. The composition of claim 38, further comprising at least one buffer and at least one salt.

40. The composition of claim 38, further comprising a ligand that binds to the isolated EBA-175 RII protein forming a complex.

41. The composition of claim 40, wherein said ligand is selected from the group consisting of N-acetylneuraminic acid, 2-deoxy-2,3-dehydro-N-acetylneuraminic acid, N-glycolyl-neuraminate, α-2,3-sialyllactose, 2-oxo-2-(4-phenoxy phenyl)ethyl 2-(3-chlorophenyl) 1,3-dioxo-5-isoindoline carboxylate, C₂₇H₂₀F₂N₂O₃, 8-(2-(3,4-dimethoxy-phenyl)-ethylamino)-3-methyl-7-phenethyl-3,7-2H-purine-2,6-dione, C₂₆H₂₀FNO₆, C₃₁H₂₄FN₃O₅S, 2′,3′-dibenzoyluridine, C₂₄H₂₁N₅O₄S, C₂₈H₁₆N₂O₆S, C₂₅H₂₄FNO₆, C₂₇H₂₅FN₂O₆S, and analogs or derivatives thereof.

42. The composition of claim 40, wherein said ligand comprises α-2,3-sialyllactose.

43. A crystal comprising the isolated EBA-175 RII protein of claim 37.

44. A co-crystal comprising the isolated EBA-175 RII protein of claim 37 complexed with one or more ligands selected from the group consisting of N-acetylneuraminic acid, 2-deoxy-2,3-dehydro-N-acetylneuraminic acid, N-glycolyl-neuraminate, α-2,3-sialyllactose, 2-oxo-2-(4-phenoxy phenyl)ethyl 2-(3-chlorophenyl)1,3-dioxo-5-isoindoline carboxylate, C₂₇H₂₀F₂N₂O₃, 8-(2-(3,4-dimethoxy-phenyl)-ethylamino)-3-methyl-7-phenethyl-3,7-2H-purine-2,6-dione, C₃₁H₂₄FN₃O₅S, 2′,3′-dibenzoyluridine, C₂₄H₂₁N₅O₄S, C₂₈H₁₆N₂O₆S, C₂₅H₂₄FNO₆, and analogs or derivatives thereof.

45. The co-crystal of claim 44, wherein said one or more ligands comprise α-2,3-sialyllactose.

46. A crystalline molecule or molecular complex having a three-dimensional structure defined by a set of atomic coordinates selected from Table 5 or Table 7, wherein said set of atomic coordinates define a first set of binding site(s) comprising amino acid residues N417, R422, N429, K439 and D442 of one monomer and K28 of the other monomer, a second set of binding site(s) comprising amino acid residues N550, N551, Y552, K553 and M554 of one monomer, and N33 of the other monomer, or a third set of binding site(s) comprising amino acid residues T340, K341, D342, V343, Y415, Q542 and Y546 of one monomer and K28, N29, R31 and S 32 of the other monomer, or a combination thereof.

47. A crystalline molecule or molecular complex characterized by a three-dimensional structure with a root mean square deviation of less than 2.5 A in backbone residue atoms from the three-dimensional structure of the crystalline molecule or molecular complex of claim 46.

48. An atomic three-dimensional structure of erythrocyte binding antigen 175 (EBA-175) region II protein defined by the set of atomic coordinates of Table 5.

49. A computer-based method for modeling the three-dimensional structure of a target protein, comprising:
(d) obtaining a three-dimensional representation of an EBA-175 RII protein or a functional domain thereof;
(e) determining similarity between amino acid sequence of the target protein and that of the EBA-175 RII protein or the functional domain thereof; and
(f) constructing a three-dimensional structural model for the target protein based on the three-dimensional representation of an EBA-175 RII protein or the functional domain thereof.

50. The method of claim 49, wherein the three-dimensional structural model for the target protein is constructed by using a comparative modeling process.

51. The method of claim 49, wherein the three-dimensional structural model for the target protein is constructed by using a threading or fold recognition process.
